SEQUENCE LISTING (6 sequences)

SEQ ID NO: 1 - DNA, 1731 bp, Artificial Sequence
  Synthetic spider silk protein
atggctagcatgactggtggacagcaaatgggtcgcggatccatgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggctaa

SEQ ID NO: 2 - protein, 576 aa, Artificial Sequence
  Synthetic Construct
MASMTGGQQMGRGSMGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPG

SEQ ID NO: 3 - DNA, 1683 bp, Artificial Sequence
  R16 Silk protein
atggctagcatgactggtggacagcaaatgggtcgcggatccatgggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggctaa

SEQ ID NO: 4 - protein, 560 aa, Artificial Sequence
  Synthetic Construct
MASMTGGQQMGRGSMGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYG

SEQ ID NO: 5 - DNA, 1923 bp, Artificial Sequence
  S16 Silk protein
atggctagcatgactggtggacagcaaatgggtcgcggatccatgggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggctaa

SEQ ID NO: 6 - protein, 640 aa, Artificial Sequence
  Synthetic Construct
MASMTGGQQMGRGSMGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYG

(19) United States
(12) Patent Application Publication
(10) Pub. No.: US 2011/0136669 A1
(43) Pub. Date: Jun. 9, 2011

(54) Continuous Fiber Layer Comprising an Active Substance on the Basis of Bio-Polymers, the use Thereof, and Method for the Production Thereof

(75) Inventors: Burghard Liebmann, Bensheim (DE); Evgueni Klimov, Ludwigshafen (DE)

(73) Assignee: BASF SE, Ludwigshafen (DE)

(21) Appl. No.: 13/058,141

(22) PCT Filed: Aug. 7, 2009
(86) PCT No.: PCT/EP09/05756

§ 371 Date: Feb. 8, 2011

(30) Foreign Application Priority Data
Aug. 8, 2008 (EP) 08162121.1
Mar. 27, 2009 (EP) 09156540.8

(51) Int. Cl. A01N 31/16; B05D 3/12; A61K 9/70; B05D 3/14; A61K 31/075; A23J 1/00

(52) U.S. Cl. 504/351; 427/240; 424/443; 427/485; 514/718; 426/656

(57) ABSTRACT

The invention relates to continuous fiber layers comprising an active substance on the basis of bio-polymers, comprising a fibrous, bio-polymer active substance carrier, and at least one active substance associated with the carrier and releasable from the continuous fiber layer; to formulations comprising an active substance, said formulations comprising such continuous fiber layers; to the use of continuous fiber layers comprising an active substance for the production of formulations comprising an active substance; and to a method for the production of continuous fiber layers comprising an active substance. The invention further relates to corresponding continuous fiber layers comprising an active substance and to the use thereof for the production of wound treatment and hygiene products, and to the respectively produced wound treatment and hygiene products.

The invention relates to active ingredient-containing fibrous sheetlike structures based on biopolymers, comprising a fibrous, biopolymeric active ingredient carrier and at least one active ingredient which is associated with the carrier and can be released by the fibrous sheetlike structure; to active ingredient-containing formulations comprising such fibrous sheetlike structures; to the use of inventive active ingredient-containing fibrous sheetlike structures for production of active ingredient-containing formulations; and to processes for production of inventive fibrous sheetlike structures. The invention further relates to corresponding active ingredient-free fibrous sheetlike structures and to the use thereof for production of wound care and hygiene articles, and to the correspondingly produced wound care and hygiene articles themselves.

### STATE OF THE ART

WO-A-2007/082936 describes the use of amphiphilic, self-assembly proteins for formulation of sparingly water-soluble active ingredients by dispersing the effect substances in a protein-containing protective colloid. After mixing the sparingly water-soluble active ingredients and the amphiphilic, self-assembly proteins in a combined disperse phase, and subsequent phase separation into a high-protein and -effect substance phase and a low-protein and -effect substance phase, protein microbeads are present, into which the sparingly water-soluble active ingredients have been encapsulated.

Various publications describe the production of fibers by spinning processes from chemically synthesized polymers and biopolymers, and also proteins.

For production of nano- and mesofibers, the person skilled in the art is aware of a multitude of processes, among which electrospinning is currently of the greatest significance. In this process, which is described, for example, by D. H. Reneker, H. D. Chun in Nanotechn. 7 (1996), pages 216 ff., a polymer melt or a polymer solution is typically exposed to a high electrical field at an edge which serves as an electrode. This can be achieved, for example, by extruding the polymer melt or polymer solution through a cannula connected to one pole of a voltage source in an electrical field under low pressure. Owing to the resulting electrostatic charging of the polymer melt or polymer solution, the result is a material flow directed toward the counterelectrode, which solidifies on the way to the counterelectrode. Depending on the electrode geometries, this process affords nonwovens or assemblies of ordered fibers.

DE-A1-10133393 discloses a process for producing hollow fibers with an internal diameter of 1 to 100 nm, in which a solution of a water-insoluble polymer—for example a poly-L-lactide solution in dichloromethane or a nylon-46 solution in pyridine—is electrospun. A similar process is also known from WO-A1-01/09414 and DE-A1-10355665.

DE-A1-19600162 discloses a process for producing lawnmower wire or textile sheetlike structures, in which polyamide, polyester or polypropylene as a thread-forming polymer, a maleic anhydride-modified polyethylene/polypropylene rubber and one or more aging stabilizers are combined, melted and mixed with one another, before this melt is melt-spun.

DE-A1-10 2004 009 887 relates to a process for producing fibers having a diameter of <50 μm by electrostatic spinning or spraying of a melt of at least one thermoplastic polymer.

The electrospinning of polymer melts can produce only fibers having diameters greater than 1 μm. For a multitude of applications, for example filtration applications, however, nano- and/or mesofibers with a diameter of less than 1 μm are required, which can be produced by the known electrospinning processes only by use of polymer solutions.

A further suitable process for producing fiber nonwovens is centrifuge spinning (also called rotor spinning). EP-B1-0624665 and EP-A1-1088918 (both BASF applications) disclose a process for producing fibrous structures from melamine-formaldehyde resin and blends thereof with thermoplastic polymers by means of centrifugal spinning processes on a spinning plate.

The process and the device for production of fibers from melts of different polymer materials with the aid of centrifugal forces are described in DE-A-102005048939.

The processing of spider silk proteins from the spider Nephila clavipes from a hexafluoro-2-propanol solution to give nanofibers by means of the electrospinning process was described in 1998 by Zarkoob and Reneker (Polymer 45: 3973-3977, 2004). Attempts to spin Bombyx mori silk from a formic acid solution are disclosed by Sukigara and Ko (Polymer 44: 5721-572, 2003), in which variation of the electrospinning parameters influences the fiber morphology. Jin and Kaplan reported water-based electrospinning of silk or silk/polyethylene oxide (Biomacromolecules 3: 1233-1239, 2002).

WO-A-03/060099 describes various methods (including electrospinning) and apparatuses for spinning Bombyx mori silk proteins and spider silk proteins. The spider silk proteins used were produced recombinantly with transgenic goats and purified from their milk and then spun.

WO-A-01/54667 describes the production of pharmaceutical compositions comprising a pharmaceutically acceptable polymeric carrier produced by electrospinning of organic polymers, such as especially polyethylene oxide, wherein a pharmaceutical agent is present in the carriers. WO 04/014304 describes corresponding pharmaceutical compositions comprising polymeric carriers, obtained by electrospinning of polyacrylates, polymethacrylates, polyvinylpyrrolidolene or polyvinylpyrrolidone or polyvinylpyrrolidone-polyvinyl acetate copolymers.

WO-A-2007/082936 describes the formulation of sparingly water-soluble effect substances with the aid of amphiphilic, self-assembly proteins. In this method, induced phase separation processes form what are called protein microbeads. However, efficient formulation of water-soluble active ingredients is not possible by this process.

The processes known to date for formulation of active ingredients and effect substances do not meet all requirements which are placed on an active ingredient formulated especially for pharmaceutical use, such as mechanical stability, nontoxicity, biocompatibility, high active ingredient bioavailability.

In addition, the active ingredients formulated by known processes are often in crystalline form, which distinctly lowers the bioavailability thereof. Especially the continuous, delayed, controlled release of the active ingredients over prolonged periods constitutes a particular challenge in the production of a suitable formulation.

Moreover, the prior art to date has not disclosed any process which is equally suitable for the formulation of a wide variety of different active ingredient classes in a polymeric carrier.

### SUMMARY OF THE INVENTION

It was therefore an object of the present invention to provide a process which allows the formulation of essentially all active ingredient classes using suitable carriers as a formulating aid, possibly while better fulfilling one or more of the abovementioned criteria than the processes known from the prior art.

In the field of active pharmaceutical ingredients, especially of the cough inducers and mucolytics of the guaiacol derivatives, there are reference products, for example tablets of the Mucinex® brand, which display continuous, delayed release profiles, for example of the active ingredient guaiacol glyceryl ether (also known as guaifenesin). However, active ingredient release is achieved here only under gastric conditions. Intestinal conditions do not lead to active ingredient release. For the most part, chemically synthesized, non-biocompatible polymers are utilized as formulating aids, which do not have any further benefit, for example an increase in the active ingredient bioavailability by enhanced absorption. It was accordingly a further object of the present invention to provide a biocompatible formulation for active cough-inducing and mucolytic ingredients, for example guaiacol glyceryl ether, which allows a continuous and delayed active ingredient release which is also triggered proteolytically, by means of proteases which occur in the gastrointestinal tract, under the conditions which exist therein.

The above objects are surprisingly achieved by provision of active ingredient-containing fibrous sheetlike structures comprising a fibrous polymeric active ingredient carrier and a releasable active ingredient associated with the carrier, wherein the carrier comprises at least one biopolymer as a polymer component.

More particularly, it is possible in accordance with the invention to utilize the proteases which occur naturally, for example, in the gastrointestinal tract, in the soil (by means of microorganisms) or on the skin as a targeted, controllable trigger mechanism for the continuous and delayed release of the active ingredients from the novel formulations described here. In addition, it is possible by the processes described here to produce active ingredient formulations in which the active ingredient is also present in amorphous form or as a solid solution. In contrast to the crystalline form, these can bring about increased active ingredient bioavailability, which can be enhanced once again in combination with the biopolymeric formulating aids, such as the amphiphilic, self-assembly proteins.

### DESCRIPTION OF FIGURES

The appended figures show:

FIG. 1 an electron microscopy (SEM) image of sheetlike C16 spider silk protein structures (fibers) with incorporated guaiacol glyceryl ether (GGE) active ingredient;

FIG. 2 crystallinity studies (WAXS in transmission) of the GGE active ingredient in the C16 spider silk protein formulations obtained by electrospinning compared to the pure substances (GGE or C16 powder);

FIG. 3 the release of the GGE active ingredient from a C16 spider silk protein formulation obtained by electrospinning and compressed to tablets in potassium phosphate buffer (control) and artificial gastric juice and intestinal juice. The 100% value was set to the total active ingredient concentration stated in the corresponding working example;

FIG. 4 the release of the GGE active ingredient from commercial tablets of the Mucinex® brand (from Adams Respiratory Therapeutics);

FIG. 5 electron microscopy (SEM) images of sheetlike C16 spider silk protein structures (fibers) with the incorporated clotrimazole active ingredient;

FIG. 6 crystallinity studies (WAXS in transmission) of the clotrimazole active ingredient in the C16 spider silk protein formulations obtained by electrospinning compared to pure clotrimazole;

FIG. 7 the release of the clotrimazole active ingredient from a C16 spider silk protein formulation which has been obtained by electrospinning and compressed to tablets in potassium phosphate buffer (control) and artificial gastric juice and intestinal juice. The 100% value was set to the active ingredient concentration stated in the corresponding example;

FIG. 8 electron microscopy (SEM) images of sheetlike C16 spider silk protein structures (fibers) with the incorporated metazachlor active ingredient;

FIG. 9 crystallinity studies (WAXS in transmission) of the metazachlor active ingredient in the C16 spider silk protein formulations obtained by electrospinning compared to pure metazachlor;

FIG. 10 the release of the metazachlor active ingredient from a C16 spider silk protein formulation obtained by electrospinning in potassium phosphate buffer (control) and proteolytically active proteinase K solution;

FIG. 11 electron microscopy (SEM) images of sheetlike C16 spider silk protein structures (fibers) with incorporated Uvinul A+ active ingredient;

FIG. 12 crystallinity studies (WAXS in transmission) of the Uvinul A+ active ingredient in the C16 spider silk protein formulations obtained by electrospinning compared to Uvinul A+ pure substance;

FIG. 13 the release of the Uvinul A+ active ingredient from a C16 spider silk protein formulation obtained by electrospinning in potassium phosphate buffer (control) and in proteolytically active proteinase K solution;

FIG. 14 light and electron microscopy (SEM) images of (A) sheetlike R16 protein structures (fibers) and (B) sheetlike S16 protein structures (fibers);

FIG. 15 electron microscopy (SEM) images of sheetlike R16 (cf. (A)) and S16 (cf. (B)) protein structures with incorporated Uvinul A+ active ingredient;

FIG. 16 crystallinity studies (WAXS in transmission) of the Uvinul A+ active ingredient in the R16 protein nonwoven (A) and S16 protein nonwoven (B) obtained by electrospinning compared to pure Uvinul A+;

FIG. 17 the release of the Uvinul A+ active ingredient from an R16 protein nonwoven (A) and S16 protein nonwoven (B) obtained by electrospinning in potassium phosphate buffer (control) and in proteolytically active proteinase K solution.

### DETAILED DESCRIPTION OF THE INVENTION

1. Definition of terms used:

Unless stated otherwise, the following definitions of technical terms apply in the context of the present description:

A “carrier polymer” is understood to mean biopolymers or blends thereof, or else blends of at least one synthetic polymer and a biopolymer, the carrier polymer having the ability to enter into noncovalent interactions with the active ingredient(s)/effect substance(s) to be formulated, or to surround or to adsorb (bear) particulate active ingredients (in dispersed or crystalline form).

A “noncovalent” interaction is understood to mean all types of bonds known to those skilled in the art which do not involve formation of covalent bonds between active ingredient and carrier polymer. Nonlimiting examples thereof include the following: hydrogen bond formation, complex formation, ionic interaction.

An “active ingredient” or “effect substance” is understood to mean synthetic or natural, low molecular weight substances with hydrophilic, lipophilic or amphiphilic properties, which can find use in agrochemistry, pharmacy, cosmetics or the foods and animal feeds industry; and likewise biological active macromolecules which can be embedded into or adsorbed onto an inventive fibrous sheetlike structure, for example peptides (such as oligopeptides having 2 to 10 amino acid residues and polypeptides having more than 10, for example 11 to 100, amino acid residues), and also enzymes and single- or double-strand nucleic acid molecules (such as oligonucleotides having 2 to 50 nucleic acid residues and polynucleotides having more than 50 nucleic acid residues).

“Low molecular weight” means molar masses of less than 5000, especially less than 2000, for example 100 to 1000, grams per mole.

“High molecular weight” means molar masses of more than 5000, especially less than 10 000 000, for example 10 000 to 1 000 000, grams per mole.

The terms “active ingredient” and “effect substance” are used synonymously.

According to the invention, the term “fibrous sheetlike structure” comprises both individual polymer fibers and the ordered or unordered single- or multilayer combination of a multitude of such fibers, for example to give fiber webs or nonwoven webs.

An “active ingredient carrier” is in fibrous form and bears, preferably in adsorbed, noncovalently bonded form on the fiber surface and/or integrated into the fiber material, the active ingredient(s) to be processed in accordance with the invention. The active ingredient may be present in homogeneous or inhomogeneous distribution over the fiber. The active ingredient may additionally be reversibly adsorbed in amorphous, semicrystalline or crystalline form on/in the active ingredient carrier.

A “soluble” active ingredient carrier is partly or fully soluble in an aqueous or organic solvent, preferably an aqueous solvent, for example water or a water-based solvent, within a pH range of pH 2 to 13, for example 4 to 11. Thus, the solubility in water can vary within a wide range—i.e. from good, i.e. rapid and complete or essentially complete solubility to very slow and complete or incomplete solubility.

Suitable “synthetic” polymeric constituents of the inventive active ingredient formulations are in principle all polymers which are soluble in water or/and in organic solvents within a temperature range between 0 and 240° C., a pressure range between 1 and 100 bar, a pH range from 0 to 14 or ionic strengths up to 10 mol/l.

An “aqueous polymer dispersion” in the context of the present invention refers, also in agreement with general technical knowledge, to a mixture of at least two mutually immiscible or essentially immiscible phases, one of the at least two phases being water, and the second comprising at least one essentially water-insoluble polymer, and especially consisting thereof. “Essentially water-insoluble polymers” in the context of the present invention are especially polymers having a solubility in water of less than 0.1% by weight, based on the total weight of the solution.

A “degradable” active ingredient carrier is present when the fiber structure is partly or completely destroyed by chemical, biological or physical processes, for example by the action of light or other radiation, solvents, chemical or biochemical oxidation, hydrolysis, proteolysis. Biochemical processes can be mediated by enzymes or microorganisms, for example by prokaryotes or eukaryotes, for example bacteria, yeasts, fungi.

“Miscibility” of polymers is understood in accordance with the invention to mean that, in the case of a mixture of at least two different synthetic polymers or biopolymers, one polymer can function as a solvent for the other. This means that a monophasic system forms between the two different polymers. In the case of immiscible components, two different phases are correspondingly present.

A “composite polymer” is understood in accordance with the invention to mean a homogeneous or inhomogeneous mixture of at least one fiber-forming polymer component with at least one low molecular weight or high molecular weight additive, such as especially a nonpolymerizable additive, for example an active ingredient or effect substance as defined above.

A “processed form” of a fibrous sheetlike structure is understood to mean that the product originally obtained in the production of the fibrous sheetlike structure is processed further; for example that the fibers are compressed or tableted, applied to a further carrier and/or subjected to a comminution to shorten the fiber length.

Unless stated otherwise, molecular weight figures for polymers relate to Mn or Mw values.

2. Preferred embodiments:

The invention firstly relates to an active ingredient-containing fibrous sheetlike structure comprising a fibrous, polymeric, soluble and/or degradable active ingredient carrier and one or more, for example 2, 3, 4 or 5, low molecular weight or high molecular weight active ingredients which are associated with the carrier and can be released by the fibrous sheetlike structure, wherein the carrier comprises, as a polymer component, one or more, for example 2, 3, 4 or 5, structure- or framework-forming, readily aggregating biopolymers, some of them of relatively high molecular weight, which may optionally additionally be modified chemically and/or enzymatically, for example by esterification, amidation, hydrolysis, carboxylation, acetylation, acylation, hydroxylation, glycosylation and farnesylation.

The fibrous sheetlike structure is especially obtainable by means of a spinning process, especially by electrospinning of an electrospinnable solution which comprises the at least one biopolymer and the at least one active ingredient, especially in dissolved form. In the fibrous sheetlike structure, the at least one active ingredient is in amorphous, semicrystalline or crystalline form.

The active ingredient is integrated (embedded) into and/or adsorbed onto the carrier.

The biopolymer is preferably a protein, especially an amphiphilic, self-assembly protein.

The amphiphilic self-assembly proteins are preferably microbead-forming proteins.

The amphiphilic self-assembly proteins are preferably intrinsically unfolded proteins.

More particularly, the amphiphilic self-assembly protein is a silk protein, for example a spider silk protein.

One example of a suitable spider silk protein is the C16 spider silk protein comprising an amino acid sequence according to SEQ ID NO: 2 or a spinnable protein derived from this protein having a sequence identity of at least about 60%, for example at least about 70, 80, 90, 95, 96, 97, 98 or 99%.

Examples of other intrinsically unfolded, amphiphilic self-assembly proteins are the R16 protein comprising an amino acid sequence according to SEQ ID NO: 4 or the S16 protein comprising an amino acid sequence according to SEQ ID NO: 6; or a spinnable protein derived from these proteins having a sequence identity of at least about 60%, for example at least about 70, 80, 90, 95, 96, 97, 98 or 99%.

More particularly, the invention provides fibrous sheetlike structures wherein at least one active pharmaceutical ingredient is present, for example an active cough-inducing and mucolytic ingredient (expectorant); such as especially the active ingredient guaiacol glyceryl ether (guaifenesin; CAS number 93-14-1) or a derivative thereof.

The invention further provides a fibrous sheetlike structure wherein the active ingredient is an active crop protection ingredient, or an active skin- and/or hair-cosmetic ingredient.

The invention further provides a fibrous sheetlike structure wherein the carrier comprises at least one further polymer component which is selected from synthetic polymers, such as especially synthetic homo- or copolymers.

The invention also provides those fibrous sheetlike structures wherein the polymeric carrier is a composite polymer which is selected from
a. mixtures of at least 2 miscible biopolymers;
b. mixtures of at least 2 immiscible biopolymers;
c. mixtures of at least one synthetic homo- or copolymer and at least one biopolymer, which are miscible with one another;
d. mixtures of at least one synthetic homo- or copolymer and at least one biopolymer, which are immiscible with one another.

In the inventive fibrous sheetlike structures, the synthetic polymer component has a molar mass (Mw) in the range from about 500 to 10 000 000, for example 1000 to 1 000 000, or 10 000 to 500 000 or 25 000 to 250 000.

The diameter of the inventive active ingredient carrier fibers is about 10 nm to 100 μm, such as 50 nm to 10 μm, or 100 nm to 2 μm. The active ingredient loading thereof is about 0.01 to 80% by weight, for example about 1 to 70% by weight or about 10 to 50% by weight, based in each case on the solids content of the fibrous sheetlike structure.

More particularly, the inventive fibrous sheetlike structure is selected from polymer fibers, polymer films and polymer nonwovens.

Inventive fibrous sheetlike structures may additionally feature noncovalent interaction of carrier polymer components and active ingredients (i.e. especially formation of a molecular solution).

The invention further relates to active ingredient-containing formulations comprising a fibrous sheetlike structure as defined above in processed form, optionally in combination with at least one further formulating aid.

For example, the fibrous sheetlike structure may be present therein in comminuted or noncomminuted form.

Moreover, the formulations may comprise fibrous sheetlike structures in compacted (compressed) form (such as tablets or capsules), in powder form or applied to a carrier substrate.

The inventive formulations are especially selected from cosmetic (especially skin- and hair-cosmetic) formulations, human and animal pharmaceutical formulations, agrochemical formulations, especially fungicidal, herbicidal, insecticidal and other crop protection formulations, and food and animal feed additives, such as food and feed supplements.

The invention further relates to the use of an active ingredient-containing fibrous sheetlike structure as defined above for production of an inventive active ingredient-containing formulation; and to the use of an active ingredient-containing formulation as defined above for controlled release of an active ingredient present therein.

Finally, the invention provides a process for producing a fibrous sheetlike structure as defined above, wherein
a. at least one active ingredient is mixed together with the at least one biopolymer component in a combined liquid phase and
b. then the embedding (adsorption) of the active ingredient into (onto) the biopolymer fiber is performed by means of spinning processes.

More particularly, the procedure therein is to mix at least one active ingredient and the polymer component in a solvent phase and to spin them from this mixture; or to mix at least one active ingredient and the polymer component in a mixture of at least two mutually miscible solvents, active ingredients and polymers being soluble at least in one of the solvents, and to spin them from this mixture.

More particularly, the invention provides a process for producing a fibrous sheetlike structure, wherein the biopolymer is an amphiphilic, self-assembly protein, which is mixed with at least one active ingredient in formic acid and then they are spun from this mixture.

The spinning process employed is preferably an electrospinning process or a centrifuge (rotor) spinning process.

The operating temperature is especially in the range from about 5 to 50° C.

The invention further relates to fibrous sheetlike structures comprising carrier material designated above, which, however, is essentially free of active ingredients, especially low molecular weight active ingredients.

The invention further provides for the use of such fibrous sheetlike structures for production of an active ingredient-containing or active ingredient-free formulation which is selected, for example, from cosmetic, human and animal pharmaceutical, agrochemical formulations, food and animal feed additives.

The invention further relates to active ingredient-free fibrous sheetlike structures comprising a fibrous, polymeric, soluble and/or degradable carrier, wherein the carrier comprises, as a polymer component, at least one biopolymer which has optionally additionally been chemically and/or enzymatically modified, and wherein the biopolymer is an amphiphilic, self-assembly protein; and wherein the biopolymer is especially a silk protein which is selected from the R16 protein comprising an amino acid sequence according to SEQ ID NO: 4, and the S16 protein comprising an amino acid sequence according to SEQ ID NO: 6; or a spinnable protein derived from these proteins having a sequence identity of at least about 60%.

The invention further provides for the use of such active ingredient-free fibrous sheetlike structures for production of medical wound treatment and wound care products and hygiene articles.

The invention also provides wound treatment and wound care products produced using an inventive fibrous sheetlike structure, for example wound dressings, plasters, tamponades, wound adhesives, bandages, bandage materials. The inventive wound materials can be used, for example, to cover the surface of minor wounds, such as cuts, or larger wounds, such as diabetic wounds, ulcers, such as pressure ulcers, surgical wounds, burns, eczema and the like. For example, inventive products can be used in the treatment of bleeding or nonbleeding wounds or injuries in the region of the skin, the eyes, the ears, the nose, the oral cavity, the teeth, and within the body, such as surgery in the intestinal region (abdomen, intestinal tract, liver, kidneys, urinary tract), thorax (heart, lungs), genital region, skull, musculature; in the treatment and care of wounds in connection with the transplantation of tissue, vessels or organs.

The invention also provides hygiene articles produced using an inventive fibrous sheetlike structure, as typically used in the personal care sector, such as diapers, incontinence products, panty liners, sanitary napkins, tampons, pads for skin and face care, wipes and the like.

3. Further configurations of the invention:

### (i) Biopolymers

Suitable in principle for formation of inventive carrier structures are those biopolymers which have the ability to form framework structures and/or to aggregate readily.

Usually, a high molecular weight is needed for this purpose, which can lead to subsequent intermolecular interloping of the molecule chains. However, intramolecular, noncovalent interactions, such as hydrogen bonds or hydrophobic interactions, can also be involved in the formation of the inventive carrier structures.

Nonlimiting examples include: cellulose, cellulose ethers, for example methyl cellulose (degree of substitution 3-40%), ethyl cellulose, butyl cellulose, hydroxymethyl celluloses; hydroxyethyl celluloses; hydroxypropyl celluloses, isopropyl cellulose, cellulose esters, for example cellulose acetate, bacterial celluloses, starches, modified starches, for example methyl ether starch, gum arabic, chitin, shellac, gelatin, chitosan, pectin, casein, alginate, and copolymers and block copolymers formed from the monomers of the abovementioned compounds; and nucleic acid molecules.

Suitable biopolymers of which particular mention should be made are amphiphilic, self-assembly proteins. Amphiphilic, self-assembly proteins consist of polypeptides formed from amino acids, especially from the 20 naturally occurring amino acids. The amino acids may also be modified, for example acetylated, glycosylated, farnesylated.

The self-assembly properties thereof enable particular proteins usable in accordance with the invention to assume higher molecular weight structures and hence to encapsulate active ingredients in a lasting manner. These amphiphilic, self-assembly proteins are suitable as formulating aids primarily for sparingly water-soluble, hydrophobic active ingredients. By virtue of their amphiphilic molecular character, these proteins interact strongly with active hydrophobic ingredients and can stabilize them in aqueous solutions. Subsequent phase separation processes can be used to encapsulate the active hydrophobic ingredients into a protein matrix. The interaction of amphiphilic, self-assembly proteins with active ingredients of greater water solubility is much weaker, which is why induced phase separation processes from aqueous solution, for example by addition of lyotropic salts, do not lead to effective encapsulation of the water-soluble active ingredients, for example in microbeads. Spinning processes can produce, from aqueous solutions or organic solvents in which amphiphilic, self-assembly proteins and water-soluble active ingredients are present in dissolved or dispersed form, higher molecular weight protein structures such as sheetlike protein structures (e.g. protein films, protein fibers, protein nonwovens). It is thus also possible to encapsulate water-insoluble or sparingly water-soluble active ingredients.

The protein- and active ingredient-rich phases produced can be cured later and removed in the form of mechanically stable active ingredient-comprising protein structures and optionally dried, and processed to tablets or capsules.

Suitable amphiphilic, self-assembly proteins for the formulation both of water-soluble and of sparingly water-soluble effect substances are those proteins which can form protein microbeads. Protein microbeads have a globular shape with a mean particle diameter of 0.1 to 100, especially of 0.5 to 20, preferably of 1 to 5 and more preferably of 2 to 4 μm.

Protein microbeads can preferably be prepared by the process described hereinafter:

The protein is dissolved in a first solvent. The solvents used may, for example, be aqueous salt solutions. Especially suitable are highly concentrated salt solutions with a concentration greater than 2, especially greater than 4 and more preferably greater than 5 molar, the ions of which have more pronounced chaotropic properties than sodium and chloride ions. One example of such a salt solution is 6 M guanidinium thiocyanate or 9 M lithium bromide. In addition, it is possible to use organic solvents to dissolve the proteins. Especially suitable are fluorinated alcohols or cyclic hydrocarbons or organic acids. Examples thereof are hexafluoroisopropanol, cyclohexane and formic acid. The protein microbeads can be produced in the solvents described. Alternatively, this solvent can be replaced by a further solvent, for example salt solutions of low concentration (c<0.5 M) by dialysis or dilution. The final concentration of the dissolved protein should be between 0.1-100 mg/ml. The temperature at which the process is performed is typically 0-80, preferably 5-50 and more preferably 10-40° C.

In the case of use of aqueous solutions, a buffer may also be added thereto, preferably in the range of pH 4-10, more preferably 5-9, most preferably 6-8.5.

Addition of an additive induces a phase separation. This forms a protein-rich phase emulsified in the mixture of solvent and additive. Due to surface effects, emulsified protein-rich droplets assume a round shape. Through the selection of the solvent, of the additive and of the protein concentration, it is possible to adjust the mean diameter of the protein microbeads to values between 0.1 μm and 100 μm.

The additives used may be all substances which are firstly miscible with the first solvent and secondly induce the formation of a protein-rich phase. When the microbead formation is performed in organic solvents, suitable substances for this purpose are organic substances which have a lower polarity than the solvent, for example toluene. In aqueous solutions, the additives used may be salts whose ions have more pronounced cosmotropic properties than sodium and chloride ions (e.g. ammonium sulfate; potassium phosphate). The final concentration of the additive should, depending on the type of additive, be between 1% and 50% by weight based on the protein solution.

The protein-rich droplets are fixed by hardening, the round shape being preserved. The fixing is based on the formation of strong intermolecular interactions. The type of interactions may be noncovalent, for example resulting from the formation of intermolecular β-sheet crystals, or covalent, for example resulting from chemical crosslinking. The hardening can be effected by the additive and/or by the addition of a further suitable substance. The hardening is effected at temperatures between 0 and 80° C., preferably between 5 and 60° C.

This further substance may be a chemical crosslinker. A chemical crosslinker is understood to mean a molecule in which at least two chemically reactive groups are bonded to one another via a linker. Examples thereof are sulfhydryl-reactive groups (e.g. maleimides, pydridyl disulfides, α-haloacetyls, vinyl sulfones, sulfatoalkyl sulfones (preferably sulfatoethyl sulfone)), amine-reactive groups (e.g. succinimidyl esters, carbodiimides, hydroxymethylphosphine, imido esters, PFP esters, aldehydes, isothiocyanates, etc.), carboxyl-reactive groups (e.g. amines etc.), hydroxyl-reactive groups (e.g. isocyanates etc.), unselective groups (e.g. aryl azides etc.) and photoactivatable groups (e.g. perfluorophenyl azide etc.). These reactive groups can form covalent linkages with amine, thiol, carboxyl or hydroxyl groups present in proteins.

The stabilized microbeads are washed with a suitable further solvent, for example water, and then dried by methods familiar to those skilled in the art, for example by lyophilization, contact drying or spray drying. The success of bead formation is checked with the aid of scanning electron microscopy.

For the production of protein microbeads, suitable proteins are those present predominantly in intrinsically unfolded form in aqueous solution. This state can be calculated, for example, by an algorithm which forms the basis of the program IUpred (http://iupred.enzim.hu/index.html; The Pairwise Energy Content Estimated from Amino Acid Composition Discriminates between Folded and Intrinsically Unstructured Proteins; Zsuzsanna Dosztányi, Veronika Csizmók, Péter Tompa and István Simon; J. Mol. Biol. (2005) 347, 827-839). A predominantly intrinsically unfolded state is assumed when a value of >0.5 is calculated by this algorithm for more than 50% of the amino acid residues (prediction type: long disorder).

### (ii) Silk Proteins

Further suitable proteins for the formulation of active ingredients by means of spinning processes are silk proteins. These are understood hereinafter to mean, in accordance with the invention, those proteins which comprise highly repetitive amino acid sequences and are stored in a liquid form in the animal, the secretion of which gives rise to fibers as a result of shearing or spinning (Craig, C. L. (1997) Evolution of arthropod silks. Annu. Rev. Entomol. 42: 231-67).

Particularly suitable proteins for the formulation of active ingredients by means of spinning processes are spider silk proteins which have been isolated in their original form from spiders.

Very particularly suitable proteins are silk proteins which have been isolated from the major ampullate gland of spiders.

Preferred silk proteins are ADF3 and ADF4 from the major ampullate gland of Araneus diadematus (Guerette et al., Science 272, 5258:112-5 (1996)).

Equally suitable proteins for the formulation of active ingredients by means of spinning processes are natural or synthetic proteins which derive from natural silk proteins and which have been produced heterologously in prokaryotic or eukaryotic expression systems using genetic engineering methods. Nonlimiting examples of prokaryotic expression organisms are Escherichia coli, Bacillus subtilis, Bacillus megaterium, Corynebacterium glutamicum inter alia. Nonlimiting examples of eukaryotic expression organisms are yeasts, such as Saccharomyces cerevisiae, Pichia pastoris inter alia, filamentous fungi such as Aspergillus niger, Aspergillus oryzae, Aspergillus nidulans, Trichoderma reesei, Acremonium chrysogenum inter alia, mammalian cells such as hela cells, COS cells, CHO cells inter alia, insect cells such as Sf9 cells, MEL cells inter alia.

Additionally suitable for the formulation of active ingredients by means of spinning processes are synthetic proteins based on repeat units from natural silk proteins. In addition to the synthetic repetitive silk protein sequences, they may additionally comprise one or more natural nonrepetitive silk protein sequences (Winkler and Kaplan, J Biotechnol 74:85-93 (2000)).

Also usable for the formulation of active ingredients by means of spinning processes are especially those synthetic spider silk proteins based on repeat units from natural spider silk proteins. In addition to the synthetic repetitive spider silk protein sequences, they may additionally comprise one or more natural nonrepetitive spider silk protein sequences.

Among the synthetic spider silk proteins, mention should preferably be made of C16 protein (Huemmerich et al. Biochemistry, 43(42):13604-13612 (2004)). This protein has the polypeptide sequence shown in SEQ ID NO: 2.

In addition to the polypeptide sequence shown in SEQ ID NO: 2, preference is also given particularly to functional equivalents, functional derivatives and salts of this sequence.

Additionally preferred for the formulation of active ingredients by means of spinning processes are synthetic proteins based on repeat units from natural silk proteins combined with sequences from insect structure proteins such as resilin (Elvin et al., 2005, Nature 437: 999-1002).

Among these combination proteins composed of silk proteins and resilins, mention should be made especially of the R16 and S16 proteins. These proteins have the polypeptide sequences shown in SEQ ID NO: 4 and SEQ ID NO: 6 respectively.

In addition to the polypeptide sequences shown in SEQ ID NO: 4 and SEQ ID NO: 6, preference is also given particularly to functional equivalents, functional derivatives and salts of these sequences.

(iii) Modified Biopolymers

“Functional equivalents” are understood in accordance with the invention especially to include mutants which have a different amino acid than that specified in at least one sequence position of the abovementioned amino acid sequences but nevertheless have the property of packaging effect substances. “Functional equivalents” thus comprise the mutants obtainable by one or more amino acid additions, substitutions, deletions and/or inversions, where the changes mentioned may occur in any sequence position provided that they lead to a mutant with the inventive profile of properties. Functional equivalence exists especially also when the reactivity patterns correspond in qualitative terms between mutant and unchanged polypeptide.

“Functional equivalents” in the above sense are also “precursors” of the polypeptides described, and “functional derivatives” and “salts” of the polypeptides.

“Precursors” are natural or synthetic precursors of the polypeptides with or without the desired biological activity.

Examples of suitable amino acid substitutions can be taken from the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The expression “salts” is understood to mean both salts of carboxyl groups and acid addition salts of amino groups of the inventive protein molecules. Salts of carboxyl groups can be prepared in a manner known per se and comprise inorganic salts, for example sodium, calcium, ammonium, iron and zinc salts, and salts with organic bases, for example amines, such as triethanolamine, arginine, lysine, piperidine and the like. Acid addition salts, for example salts with mineral acids, such as hydrochloric acid or sulfuric acid, and salts with organic acids, such as acetic acid and oxalic acid, likewise form part of the subject matter of the invention.

“Functional derivatives” of inventive polypeptides can likewise be prepared on functional amino acid side groups or on the N- or C-terminal end thereof with the aid of known techniques. Such derivatives comprise, for example, aliphatic esters of carboxylic acid groups, amides of carboxylic acid groups, obtainable by reaction with ammonia or with a primary or secondary amine; N-acyl derivatives of free amino groups, prepared by reaction with acyl groups; or O-acyl derivatives of free hydroxyl groups, prepared by reaction with acyl groups.

“Functional equivalents” also encompassed in accordance with the invention are homologs to the proteins/polypeptides disclosed specifically herein. These have at least 60%, for example 70, 80 or 85%, for example 90, 91, 92, 93, 94, 95, 96, 97, 98 or 99%, identity to one of the amino acid sequences disclosed specifically.

“Identity” between two sequences is understood especially to mean the identity of the radicals over the overall sequence length in each case, especially the identity which is calculated by comparison with the aid of the Vector NTI Suite 7.1 (Vector NTI Advance 10.3.0, Invitrogen Corp.) (or software from Informax (USA) using the clustal method (Higgins D G, Sharp P M. Fast and sensitive multiple sequence alignments on a microcomputer. Comput Appl. Biosci. 1989 Apr. 5(2):151-1)) with the following parameter settings:

Multiple alignment parameter:

| | |
|---|---|
| Gap opening penalty | 10 |
| Gap extension penalty | 0.05 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise alignment parameter:

| FAST algorithm | off |
|---|---|
| K-tuple size | 1 |
| Gap penalty | 3 |
| Window size | 5 |
| Number of best diagonals | 5 |

### (iv) Formulation of Active Ingredients

Formulations of active ingredients can be produced, for example, using a biopolymer such as an amphiphilic self-assembly protein in various ways. Active ingredients can be packaged or encapsulated in sheetlike protein structures (e.g. protein films, protein fibers, protein nonwovens) by spinning processes.

The fibers and sheetlike structures composed of protein-active ingredient combinations can be produced from solution or finely divided dispersion (dry spinning, wet spinning) and gel by all spinning processes known to those skilled in the art. Particularly suitable spinning processes are those from solution or a finely divided dispersion, more preferably including centrifuge spinning (rotor spinning) and electrospinning (electrostatic spinning).

In the case of spinning of proteins to fibers, suitable fiber diameters are from 10 nm to 100 μm, preferably diameters from 50 nm to 10 μm; more preferably from 100 nm to 2 μm.

In the case of electrospinning (electrostatic spinning), the solution or finely divided dispersion to be formulated is introduced into an electrical field of strength between 0.01 and 10 kV/cm, more preferably between 1 and 6 kV/cm and most preferably between 2 and 4 kV/cm. As soon as the electrical forces exceed the surface tension of the formulation, mass is transferred in the form of a jet to the opposite electrode. The solvent evaporates in the space between the electrodes, and solids in the formulation are then present in the form of fibers on the counterelectrode. The spinning electrode may be die- or syringe-based or have roller geometry. The spinning can be effected in either vertical direction (from the bottom upward and from the top downward), and in horizontal direction.

A further suitable process is centrifuge spinning (rotor spinning). In this process, the formulation or finely divided dispersion is introduced into a field with gravitational forces. For this purpose, the fiber raw material is introduced into a vessel and the vessel is set to rotate, in the course of which the fluidized fiber raw material is discharged from the vessel in the form of fibers by centripetal or centrifugal forces. The fibers can subsequently be transported away by gas flow and combined to form sheetlike structures.

The active ingredients can be formulated by inclusion into the sheetlike protein structures produced by the processes according to the invention (for example protein films, protein fibers, protein nonwovens). This process comprises two steps. In the first step, a spinning solution is prepared from active ingredient and biopolymer, for example amphiphilic self-assembly protein, by mixing the components in a common phase. For this purpose, the active ingredient and the protein can be brought into solution directly by means of a solvent or a solvent mixture. Alternatively, the active ingredient and the protein can first be dissolved in different solvents and the solutions can then be mixed with one another, so as again to give rise to a common phase. The common phase may also be a molecularly disperse phase or a colloidally disperse phase.

Further substances may additionally be added to the spinning solution, in order, for example, to increase the viscosity of the solution or to improve the processability thereof in other ways or to achieve preferred structural material properties, for example crystallinities, or preferred performance properties, for example controlled, delayed or continuous release profiles of the formulated active ingredients. Preferred additives are water-soluble polymers or especially aqueous polymer dispersions. Suitable amounts of the additives in the spinning solution are >0.1% by weight, preferably >0.5% by weight, more preferably >1% by weight, most preferably >5% by weight.

In addition, it is possible to add to the spinning solution or to the sheetlike protein structures produced therefrom (for example protein films, protein fibers, protein nonwovens) substances which enable disintegration of the tablets or capsules and hence improved dispersion of the sheetlike protein structures compressed to the tablets or capsules (for example protein films, protein fibers, protein nonwovens) and of the active ingredients present therein.

The dissolution of the active ingredient and of the protein in different solvents and the subsequent mixing of the two solutions are advantageous especially when the active ingredient and the protein cannot be dissolved in a common solvent or solvent mixture. In this way, it is also possible to produce colloidally disperse solutions of hydrophobic active ingredients, by diluting the active ingredient dissolved in a suitable solvent in another solvent in which this active ingredient is insoluble.

Since proteins generally have good water solubility, preference is given to working with aqueous solutions. However, mixtures of water and water-miscible organic solvents or the exclusive use of organic solvents are also possible. Examples of suitable water-miscible solvents are alcohols such as methanol, ethanol and isopropanol, fluorinated alcohols such as hexafluoroisopropanol and trifluoroethanol, alkanones such as acetone, or else sulfoxides, for example dimethyl sulfoxide, or formamides such as dimethylformamide, or other organic solvents, for example tetrahydrofuran and acetonitrile or N-methyl-2-pyrrolidone or formate. In general, it is possible to work with all solvents and solvent mixtures in which the proteins can be dissolved. Examples of suitable solvents are water or water-based buffer systems and salt solutions, fluorinated alcohols, for example hexafluoroisopropanol or trifluoroethanol, ionic liquids, for example 1-ethyl-3-methylimidazolium (EMIM) acetate, aqueous solutions of chaotropic salts, for example urea, guanidiunium hydrochloride and guanidinium thiocyanate, or organic acids, for example formic acid, and mixtures of these solvents with other organic solvents. Examples of solvents which can be mixed with the solvents for the protein include water, alcohols such as methanol, ethanol and isopropanol, alkanones such as acetone, sulfoxides, for example dimethyl sulfoxide, formamides such as dimethylformamide, haloalkanes such as methylene chloride, or else further organic solvents, for example tetrahydrofuran.

The second step of the formulation of the active ingredients is an assembly of the protein, induced, for example, by evaporation of the solvent, an electrical field, by shear forces or centrifugal forces, to give a combined solid or high-viscosity, gel-like phase which subsequently hardens. This incorporates the active ingredient into the assembly form of the protein. The assembled protein structures can be produced as active ingredient-containing sheetlike protein structures (for example protein films, protein fibers, protein nonwovens) and laid during the spinning operation onto substrates, for example microfiber nonwovens. Subsequently, the assembled protein structures can be compressed to tablets or capsules.

The active ingredient can be bonded to the surface, incorporated into the sheetlike protein structures (for example protein films, protein fibers, protein nonwovens), or else associated with the sheetlike protein structures in both ways. The binding of the active ingredient to the sheetlike protein structures produced by the processes according to the invention can be determined by the depletion of dissolved active ingredient in the assembly mixture. The concentration of the active ingredient can be measured by a quantitative analysis of its properties. For example, the binding of light-absorbing active ingredients can be analyzed by photometric methods. For this purpose, for example, the color of the sheetlike protein structures (for example protein films, protein fibers, protein nonwovens) or the decolorization of the low-protein and -active ingredient phase of the formulation mixture are determined by measuring the absorption of a colored or light-absorbing active ingredient. With the aid of these methods, it is also possible to determine how high the active ingredient content is in the microbeads. For this purpose, the sheetlike protein structures (for example protein films, protein fibers, protein nonwovens) are admixed with a solvent suitable for the encapsulated active ingredient, which leaches out the active ingredient. Subsequently, the active ingredient content is determined in the solvent, for example by absorption photometry. Alternatively, the protein assembly structure can also be degraded by means of proteolytically active enzymes, the active ingredient present being released and subsequently quantified.

### (v) Synthetic Polymer Components

Suitable synthetic polymers are, for example, selected from the group consisting of homo- and copolymers of aromatic vinyl compounds, homo- and copolymers of alkyl acrylates, homo- and copolymers of alkyl methacrylates, homo- and copolymers of α-olefins, homo- and copolymers of aliphatic dienes, homo- and copolymers of vinyl halides, homo- and copolymers of vinyl acetates, homo- and copolymers of acrylonitriles, and copolymers of urethanes, homo- and copolymers of vinylamides and copolymers formed from two or more of the monomer units forming the aforementioned polymers.

Useful carrier polymers include more particularly polymers based on the following monomers:
acrylamide, adipic acid, allyl methacrylate, alpha-methylstyrene, butadiene, butanediol, butanediol dimethacrylate, butanediol divinyl ether, butanediol dimethacrylate, butanediol monoacrylate, butanediol monomethacrylate, butanediol monovinyl ether, butyl acrylate, butyl methacrylate, cyclohexyl vinyl ether, diethylene glycol divinyl ether, diethylene glycol monovinyl ether, ethyl acrylate, ethyldiglycol acrylate, ethylene, ethylene glycol butyl vinyl ether, ethylene glycol dimethacrylate, ethylene glycol divinyl ether, ethylhexyl acrylate, ethylhexyl methacrylate, ethyl methacrylate, ethyl vinyl ether, glycidyl methacrylate, hexanediol divinyl ether, hexanediol monovinyl ether, isobutene, isobutyl acrylate, isobutyl methacrylate, isoprene, isopropylacrylamide, methyl acrylate, methylenebisacrylamide, methyl methacrylate, methyl vinyl ether, n-butyl vinyl ether, N-methyl-N-vinylacetamide, N-vinylcaprolactam, N-vinylimidazole, N-vinylpiperidone, N-vinylpyrrolidone, octadecyl vinyl ether, phenoxyethyl acrylate, polytetrahydrofuran 2 divinyl ether, propylene, styrene, terephthalic acid, tert-butylacrylamide, tert-butyl acrylate, tert-butyl methacrylate, tetraethylene glycol divinyl ether, triethylene glycol dimethyl acrylate, triethylene glycol divinyl ether, triethylene glycol divinyl methyl ether, trimethylolpropane trimethacrylates, trimethylolpropane trivinyl ether, vinyl 2-ethylhexyl ether, vinyl 4-tert-butylbenzoate, vinyl acetate, vinyl chloride, vinyl dodecyl ether, vinylidene chloride, vinyl isobutyl ether, vinyl isopropyl ether, vinyl propyl ether and vinyl tert-butyl ether.

The term “synthetic polymers” comprises both homopolymers and copolymers. Useful copolymers are not only random but also alternating systems, block copolymers or graft copolymers. The term “copolymers” comprises polymers formed from two or more different monomers, or else where the incorporation of at least one monomer into the polymer chain can be realized in various ways, as is the case with stereoblock copolymers for example.

It is also possible to use blends of homo- and copolymers. The homo- and copolymers may or may not be miscible with each other.

The following polymers should be mentioned with preference:
polyvinyl ethers, for example polybenzyloxyethylene, polyvinyl acetals, polyvinyl esters, for example polyvinyl acetate, polyoxytetramethylene, polyamides, polycarbonates, polyesters, polysiloxanes, polyurethanes, polyacrylamides, for example poly(N-isopropylacrylamide), polymethacrylamides, polyhydroxybutyrates, polyvinyl alcohols, acetylated polyvinyl alcohols, polyvinylformamide, polyvinylamines, polycarboxylic acids (polyacrylic acid, polymethacrylic acid), polyacrylamide, polyitaconic acid, poly(2-hydroxyethyl acrylate), poly(N-isopropylacrylamide), polysulfonic acid (poly(2-acrylamido-2-methyl-1-propanesulfonic acid) or PAMPS), polymethacrylamide, polyalkylene oxides, e.g. polyethylene oxides; poly-N-vinylpyrrolidone; maleic acids, poly(ethyleneimine), polystyrenesulfonic acid, polyacrylates, e.g. polyphenoxyethyl acrylate, polymethyl acrylate, polyethyl acrylate, polydodecyl acrylate, poly(ibornyl acrylate), poly(n-butyl acrylate), poly(t-butyl acrylate), polycyclohexyl acrylate, poly(2-ethylhexyl acrylate), polyhydroxypropyl acrylate, polymethacrylates, e.g. polymethyl methacrylate, poly(n-amyl methacrylate), poly(n-butyl methacrylate), polyethyl methacrylate, poly(hydroxypropyl methacrylate), polycyclohexyl methacrylate, poly(2-ethylhexyl methacrylate), polylauryl methacrylate, poly(t-butyl methacrylate), polybenzyl methacrylate, poly(ibornyl methacrylate), polyglycidyl methacrylate and polystearyl methacrylate, polystyrene, and also copolymers based on styrene, for example with maleic anhydride, styrene-butadiene copolymers, methyl methacrylate-styrene copolymers, N-vinylpyrrolidone copolymers, polycaprolactones, polycaprolactams, poly(N-vinylcaprolactam).

Particular mention should be made of poly-N-vinylpyrrolidone, polymethyl methacrylate, acrylate-styrene copolymers, polyvinyl alcohol, polyvinyl acetate, polyamide and polyester.

It is additionally possible to use synthetic biodegradable polymers.

The term “biodegradable polymers” shall comprise all polymers that meet the biodegradability definition given in DIN V 54900, more particularly compostable polyesters.

The general meaning of biodegradability is that the polymers, such as polyesters for example, decompose within an appropriate and verifiable interval. Degradation may be effected hydrolytically and/or oxidatively and predominantly through the action of microorganisms, such as bacteria, yeasts, fungi and algae. Biodegradability can be quantified, for example, by polyesters being mixed with compost and stored for a certain time. According to ASTM D 5338, ASTM D 6400 and DIN V 54900 CO₂-free air is, for example, flowed through ripened compost during composting and the ripened compost subjected to a defined temperature program. Biodegradability here is defined via the ratio of the net CO₂ released by the sample (after deduction of the CO₂ released by the compost without sample) to the maximum amount of CO₂ releasable by the sample (calculated from the carbon content of the sample). Biodegradable polyesters typically show clear signs of degradation, such as fungal growth, cracking and holing, after just a few days of composting. Examples of biodegradable polymers are biodegradable polyesters, for example polylactide, polycaprolactone, polyalkylene adipate terephthalates, polyhydroxyalkanoates (polyhydroxybutyrate) and polylactide glycoside. Particular preference is given to biodegradable polyalkylene adipate terephthalates, preferably polybutylene adipate terephthalates. Suitable polyalkylene adipate terephthalates are described for example in DE 4 440 858 (and are commercially available, e.g., Ecoflex® from BASF).

### (vi) Active Ingredients

The terms “active ingredients” and “effect substances” are used synonymously hereinafter. These include both water-soluble and sparingly water-soluble effect substances. The terms “sparingly water-soluble” and “hydrophobic” active ingredients or effect substances are used synonymously. Sparingly water-soluble active ingredients refer hereinafter to those compounds whose water solubility at 20° C. is <1% by weight, preferably <0.5% by weight, more preferably <0.25% by weight, most preferably <0.1% by weight. Water-soluble active ingredients refer hereinafter to those compounds whose water solubility at 20° C. is >1% by weight, preferably >10% by weight, more preferably >40% by weight, most preferably >70% by weight.

Suitable effect substances are dyes, especially those specified in the following table:

Particularly advantageous dyes are the oil-soluble or oil-dispersible compounds specified in the following list. The color index numbers (CIN) are taken from the Rowe Colour Index, 3rd edition, Society of Dyers and Colourists, Bradford, England, 1971.

| Chemical or other name | CIN | Color |
|---|---|---|
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| N-(5-Chloro-2,4-dimethoxyphenyl)-4-[[5-[(diethylamino)sulfonyl]- | 12490 | red |
| 2-methoxyphenyl]azo]-3-hydroxy-2-naphthalenecarboxamide | | |
| Pigment Yellow 16 | 20040 | yellow |
| Pigment Yellow 13 | 21100 | yellow |
| Pigment Yellow 83 | 21108 | yellow |
| Solvent Yellow | 21230 | yellow |
| Food Yellow | 40800 | orange |
| trans-β-Apo-8′-carotinaldehyde (C30) | 40820 | orange |
| trans-Apo-8′-carotinic acid (C30) ethyl ester | 40825 | orange |
| Canthaxanthin | 40850 | orange |
| Solvent Dye | 45396 | orange |
| Quinophthalone | 47000 | yellow |
| Pigment Violet 23 | 51319 | violet |
| 1,2-Dihydroxyanthraquinone, calcium-aluminum complex | 58000 | red |
| 1-Hydroxy-4-N-phenylaminoanthraquinone | 60724 | violet |
| 1-Hydroxy-4-(4′-methylphenylamino)anthraquinone | 60725 | violet |
| 1,4-Di(4′-methylphenylamino)anthraquinone | 61565 | green |
| N,N′-Dihydro-1,2,1′,2′-anthraquinonazine | 69800 | blue |
| Vat Blue 6; Pigment Blue 64 | 69825 | blue |
| Vat Orange 7 | 71105 | orange |
| Indigo | 73000 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanine | 74260 | green |
| Bixin, Nor-Bixin | 75120 | orange |
| Lycopene | 75125 | yellow |
| trans-alpha-, -beta- or -gamma-carotene | 75130 | orange |
| Keto and/or hydroxyl derivatives of carotene | 75135 | yellow |
| 1,7-Bis(4-hydroxy-3-methoxyphenyl)-1,6-heptadiene-3,5-dione | 75300 | yellow |

Further preferred effect substances are fatty acids, especially saturated fatty acids which bear an alkyl branch, more preferably branched eicosanoic acids such as 18-methyleicosanoic acid.

Further preferred effect substances are carotenoids. Carotenoids are understood in accordance with the invention to mean the following compounds, and the esterified or glycosylated derivatives thereof: β-carotene, lycopene, lutein, astaxanthin, zeaxanthin, cryptoxanthin, citranaxanthin, canthaxanthin, bixin, β-apo-4-carotenal, β-apo-8-carotenal, β-apo-8-carotinic ester, neurosporene, echinenone, adonirubin, violaxanthin, torulene, torularhodin, individually or as a mixture. Carotenoids used with preference are β-carotene, lycopene, lutein, astaxanthin, zeaxanthin, citranaxanthin and canthaxanthin.

Further preferred effect substances are vitamins, especially retinoids and esters thereof.

In the context of the present invention, retinoids mean vitamin A alcohol (retinol) and derivatives thereof, such as vitamin A aldehyde (retinal), vitamin A acid (retinoic acid) and vitamin A esters (e.g. retinyl acetate, retinyl propionate and retinyl palmitate). The term “retinoic acid” comprises not only all-trans retinoic acid but also 13-cis retinoic acid. The terms “retinol” and “retinal” preferably comprise the all-trans compounds. A preferred retinoid used for the inventive formulations is all-trans-retinol, referred to hereinafter as retinol.

Further preferred effect substances are vitamins, provitamins and vitamin precursors from groups A, B, C, E and F, especially 3,4-didehydroretinol, β-carotene (provitamin of vitamin A), palmitic esters of ascorbic acid, tocopherols, especially α-tocopherol and esters thereof, for example the acetate, the nicotinate, the phosphate and the succinate; and also vitamin F, which is understood to mean essential fatty acids, particularly linoleic acid, linolenic acid and arachidonic acid.

Further preferred effect substances are lipophilic, oil-soluble antioxidants from the group of vitamin E, i.e. tocopherol and derivatives thereof, gallic esters, flavonoids and carotenoids, and also butylhydroxytoluene/anisole.

A further preferred effect substance is lipoic acid and suitable derivatives (salts, esters, sugars, nucleotides, nucleosides, peptides and lipids).

Further preferred effect substances are UV light protection filters. This is understood to mean organic substances which are capable of absorbing ultraviolet rays and of releasing the energy absorbed again in the form of longer-wave radiation, for example heat.

The oil-soluble UV-B filters used may, for example, be the following substances:
3-benzylidenecamphor and derivatives thereof, e.g. 3-(4-methylbenzylidene)camphor; 4-aminobenzoic acid derivatives, preferably 2-ethylhexyl 4-(dimethylamino)benzoate, 2-octyl 4-(dimethylamino)benzoate and amyl 4-(dimethylamino)benzoate; esters of cinnamic acid, preferably 2-ethylhexyl 4-methoxycinnamate, propyl 4-methoxycinnamate, isoamyl 4-methoxycinnamate, isopentyl 4-methoxycinnamate, 2-ethylhexyl 2-cyano-3-phenylcinnamate (octocrylene);
esters of salicylic acid, preferably 2-ethylhexyl salicylate, 4-isopropylbenzyl salicylate, homomenthyl salicylate; derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably di-2-ethylhexyl 4-methoxybenzmalonate; triazine derivatives, for example 2,4,6-trianilino-(p-carbo-2′-ethyl-1′-hexyloxy)-1,3,5-triazine (octyltriazone) and Dioctyl Butamido Triazone (Uvasorb® HEB):
propane-1,3-diones, for example 1-(4-tert-butylphenyl)-3-(4′-methoxyphenyl)propane-1,3-dione.

Particular preference is given to the use of esters of cinnamic acid, preferably 2-ethylhexyl 4-methoxycinnamate, isopentyl 4-methoxycinnamate, 2-ethylhexyl 2-cyano-3-phenylcinnamate (octocrylene).

Additionally preferred is the use of derivatives of benzophenone, especially 2-hydroxy-4-methoxybenzophenone, 2-hydroxy-4-methoxy-4′-methylbenzophenone, 2,2′-dihydroxy-4-methoxybenzophenone, and the use of propane-1,3-diones, for example 1-(4-tert-butylphenyl)-3-(4′-methoxyphenyl)propane-1,3-dione.

Useful typical UV-A filters include:
derivatives of benzoyl methane, for example 1-(4′-tert-butylphenyl)-3-(4′-methoxy-phenyl)propane-1,3-dione, 4-tert-butyl-4′-methoxydibenzoylmethane or 1-phenyl-3-(4′-isopropylphenyl)propane-1,3-dione;
amino-hydroxyl-substituted derivatives of benzophenones, for example N,N-diethylaminohydroxybenzoyl n-hexyl benzoate.

The UV-A and UV-B filters may of course also be used in mixtures.

Suitable UV filter substances are specified in the following table:

| | | CAS No. |
|---|---|---|
| No. | Substance | (=acid) |
| 1 | 4-aminobenzoic acid | 150-13-0 |
| 2 | 3-(4′-trimethylammonium)benzylidenebornan-2-one methylsulfate | 52793-97-2 |
| 3 | 3,3,5-trimethylcyclohexyl salicylate (homosalate) | 118-56-9 |
| 4 | 2-hydroxy-4-methoxybenzophenone (oxybenzone) | 131-57-7 |
| 5 | 2-phenylbenzimidazole-5-sulfonic acid and the potassium, | 27503-81-7 |
| | sodium and triethanolamine salts thereof | |
| 6 | 3,3′-(1,4-phenylenedimethine)bis(7,7-dimethyl-2-oxo- | 90457-82-2 |
| | bicyclo[2.2.1]heptane-1-methanesulfonic acid) and salts thereof | |
| 7 | polyethoxyethyl 4-bis(polyethoxy)aminobenzoate | 113010-52-9 |
| 8 | 2-ethylhexyl 4-dimethylaminobenzoate | 21245-02-3 |
| 9 | 2-ethylhexyl salicylate | 118-60-5 |
| 10 | 2-isoamyl 4-methoxycinnamate | 71617-10-2 |
| 11 | 2-ethylhexyl 4-methoxycinnamate | 5466-77-3 |
| 12 | 2-hydroxy-4-methoxybenzophenone-5-sulfonic acid | 4065-45-6 |
| | (sulisobenzone) and the sodium salt | |
| 13 | 3-(4′-sulfobenzylidene)bornan-2-one and salts | 58030-58-6 |
| 14 | 3-benzylidenebornan-2-one | 16087-24-8 |
| 15 | 1-(4′-isopropylphenyl)-3-phenylpropane-1,3-dione | 63260-25-9 |
| 16 | 4-isopropylbenzyl salicylate | 94134-93-7 |
| 17 | 3-imidazol-4-ylacrylic acid and the ethyl ester thereof | 104-98-3 |
| 18 | ethyl 2-cyano-3,3-diphenylacrylate | 5232-99-5 |
| 19 | 2′-ethylhexyl 2-cyano-3,3-diphenylacrylate | 6197-30-4 |
| 20 | menthyl o-aminobenzoate or 5-methyl-2-(1-methylethyl)-2- | 134-09-8 |
| | aminobenzoate | |
| 21 | glyceryl p-aminobenzoate or 1-glyceryl 4-aminobenzoate | 136-44-7 |
| 22 | 2,2′-dihydroxy-4-methoxybenzophenone (dioxybenzone) | 131-53-3 |
| 23 | 2-hydroxy-4-methoxy-4-methylbenzophenone (mexenone) | 1641-17-4 |
| 24 | triethanolamine salicylate | 2174-16-5 |
| 25 | dimethoxyphenylglyoxalic acid or sodium 3,4- | 4732-70-1 |
| | dimethoxyphenylglyoxalate | |
| 26 | 3-(4′-sulfobenzylidene)bornan-2-one and salts thereof | 56039-58-8 |
| 27 | 4-tert-butyl-4′-methoxydibenzoylmethane | 70356-09-1 |
| 28 | 2,2′,4,4′-tetrahydroxybenzophenone | 131-55-5 |
| 29 | 2,2′-methylenebis[6-(2H-benzotriazol-2-yl)-(1,1,3,3,-tetra- | 103597-45-1 |
| | methylbutyl)phenol] | |
| 30 | 2,2′-(1,4-phenylene)bis-1H-benzimidazole-4,6-disulfonic acid, | 180898-37-7 |
| | sodium salt | |
| 31 | 2,4-bis[4-(2-ethylhexyloxy)-2-hydroxy]phenyl-6-(4-methoxy- | 187393-00-6 |
| | phenyl)-(1,3,5)-triazine | |
| 32 | 3-(4-methylbenzylidene)camphor | 36861-47-9 |
| 33 | polyethoxylethyl 4-bis(polyethoxy)paraaminobenzoate | 113010-52-9 |
| 34 | 2,4-dihydroxybenzophenone | 131-56-6 |
| 35 | 2,2′-dihydroxy-4,4′-dimethoxybenzophenone-5,5′-disodium | 3121-60-6 |
| | sulfonate | |
| 36 | benzoic acid 2-[4-(diethylamino)-2-hydroxybenzoyl]hexyl ester | 302776-68-7 |
| 37 | 2-(2H-benzotriazol-2-yl)-4-methyl-6-[2-methyl-3-[1,3,3,3-tetra- | 155633-54-8 |
| | methyl-1-[(trimethylsilyl)oxy]disiloxanyl]propyl]phenol | |
| 38 | 1,1-[(2,2′-dimethylpropoxy)carbonyl]-4,4-diphenyl- | 363602-15-7 |
| | 1,3-butadiene | |

In addition to the two aforementioned groups of primary light stabilizers, it is also possible to use secondary light stabilizers of the antioxidant type, which stop the photochemical reaction chain which is triggered when UV radiation penetrates into the skin. Typical examples thereof are tocopherols (vitamin E) and oil-soluble ascorbic acid derivatives (vitamin C).

According to the invention, it is possible to use suitable derivatives (salts, esters, sugars, nucleotides, nucleosides, peptides and lipids) of the compounds mentioned as effect substances.

Further preferred are what are called peroxide decomposers, i.e. compounds which are capable of decomposing peroxides, more preferably lipid peroxides. These are understood to mean organic substances, for example 5-pyrimidinol derivatives and 3-pyridinol derivatives and probucol.

In addition, the peroxide decomposers mentioned are preferably the substances described in patent applications WO-A-02/07698 and WO-A03/059312, the content of which is hereby explicitly incorporated by reference, preferably the boron-comprising or nitrogen-comprising compounds described therein, which can reduce peroxides or hydroperoxides to the corresponding alcohols without forming free-radical conversion stages. In addition, it is possible to use sterically hindered amines for this purpose.

A further group is that of antiirritants, which have an inflammation-inhibiting action on skin damaged by UV light. Such substances are, for example, bisabolol, phytol and phytantriol.

A further group of effect substances is that of active ingredients which can be used in crop protection, for example herbicides, insecticides and fungicides.

The following list of insecticides shows possible active crop protection ingredients, but no restriction thereto is intended:
A.1. organo(thio)phosphates: azinphos-methyl, chlorpyrifos, chlorpyrifos-methyl, chlorfenvinphos, diazinon, disulfoton, ethion, fenitrothion, fenthion, isoxathion, malathion, methidathion, methyl-parathion, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
A.2. carbamates: alanycarb, bendiocarb, benfuracarb, carbaryl, carbofuran, carbosulfan, fenoxycarb, furathiocarb, methiocarb, methomyl, oxamyl, pirimicarb, thiodicarb, triazamate;
A.3. pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin;
A.4. growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, a tetronic acid derivative of formula D1

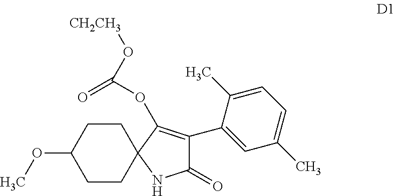

A.5. nicotine receptor agonists/antagonists: clothianidin, dinotefuran, thiacloprid;
A.6. GABA antagonists: acetoprole, endosulfan, ethiprole, fipronil, vaniliprole;
A.7. macrolide insecticides: abamectin, emamectin, milbemectin, lepimectin, spinosad;
A.8. METI I acaricides: fenazaquin, pyridaben, tebufenpyrad, tolfenpyrad;
A.9. METI II and III compounds: acequinocyl, fluacyprim, hydramethylnon;
A.10. uncoupler compounds: chlorfenapyr;
A.11. inhibitors of oxidative phosphorylation: cyhexatin, diafenthiuron, fenbutatin oxide, propargite;
A.12. ecdysone antagonists: cryomazine;
A.13. inhibitors of the mixed function oxidase: piperonyl butoxide;
A.14. sodium channel blockers: indoxacarb, metaflumizone;
A.15. various: benclothiaz, bifenazate, flonicamid, pyridalyl, pymetrozine, sulfur, thiocyclam and aminoisothiazole comnounds of the formula D2

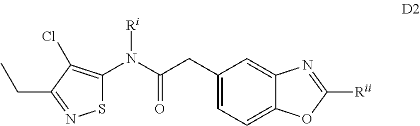

where Rⁱ is —CH₂OCH₂CH₃ or H and Rⁱⁱ is CF₂CF₂CF₃ or CH₂CH(CH₃)₃, anthranilamide compounds of the formula D3

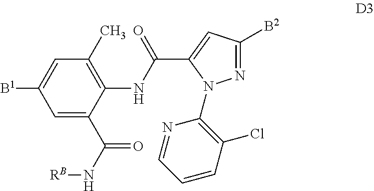

where B¹ is hydrogen or chlorine, B² is bromine or CF₃ and R⁸ is CH₃ or CH(CH₃)₂, and malononitrile compounds as described in JP 2002 284608, WO 02/189579, WO 02/190320, WO 02/190321, WO 04/106677, WO 04/120399 or JP 2004 99597, N-R′-2,2-dihalo-1-R″-cyclopropanecarboxamide-2-(2,6-dichloro-α,α,α,α-trifluoro-p-tolyl)hydrazone or N-R′-2,2-di(R″′)propionamide-2(2,6-dichloro-α,α,α,α-trifluoro-p-tolyl)hydrazone in which R′ is methyl or ethyl, halo is chlorine or bromine, R″ is hydrogen or methyl and R″′ is methyl or ethyl.

The list of fungicides below shows possible active ingredients, but no restriction thereto is intended:

### 1. Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

### 2. Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoro-2-methyltriazole-5-carboxamide, N-(4′-chloro-3′-fluorobiphenyl-2-yl)-4-difluoro-2-methyltriazole-N-(3′,4′-dichloro-4-fluorobiphenyl-2-yl)-3-difluoro-1-methylpyrazole-4-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### 3. Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### 4. Nitrogen-Containing Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### 5. Carbamates And Dithiocarbamates

carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### 6. Other Fungicides

organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
others: spiroxamine, cyflufenamid, cymoxanil, metrafenone.

The list of herbicides below shows possible active ingredients, but no restriction thereto is intended:
compounds which inhibit the biosynthesis of lipids, for example chlorazifop, clodinafop, clofop, cyhalofop, ciclofop, fenoxaprop, fenoxaprop-p, fenthiaprop, fluazifop, fluazifop-P, haloxyfop, haloxyfop-P, isoxapyrifop, metamifop, propaquizafop, quizalofop, quizalofop-P, trifop, or esters thereof, butroxydim, cycloxydim, profoxydim, sethoxydim, tepraloxydim, tralkoxydim, butylate, cycloate, diallate, dimepiperate, EPTC, esprocarb, ethiolate, isopolinate, methiobencarb, molinate, orbencarb, pebulate, prosulfocarb, sulfallate, thiobencarb, thiocarbazil, triallate, vernolate, benfuresate, ethofumesate and bensulide;
ALS inhibitors such as amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac; if the pH is <8;
compounds which inhibit photosynthesis, such as atraton, atrazine, ametryne, aziprotryne, cyanazine, cyanatryn, chlorazine, cyprazine, desmetryne, dimethametryne, dipropetryn, eglinazine, ipazine, mesoprazine, methometon, methoprotryne, procyazine, proglinazine, prometon, prometryne, propazine, sebuthylazine, secbumeton, simazine, simeton, simetryne, terbumeton, terbuthylazine and terbutryne;
protoporphyrinogen-IX oxidase inhibitors such as acifluorfen, bifenox, chlomethoxyfen, chlornitrofen, ethoxyfen, fluorodifen, fluoroglycofen, fluoronitrofen, fomesafen, furyloxyfen, halosafen, lactofen, nitrofen, nitrofluorfen, oxyfluorfen, fluazolate, pyraflufen, cinidon-ethyl, flumiclorac, flumioxazin, flumipropyn, fluthiacet, thidiazimin, oxadiazon, oxadiargyl, azafenidin, carfentrazone, sulfentrazone, pentoxazone, benzfendizone, butafenacil, pyraclonil, profluazol, flufenpyr, flupropacil, nipyraclofen and etnipromid;
herbicides such as metflurazon, norflurazon, flufenican, diflufenican, picolinafen, beflubutamid, fluridone, flurochloridone, flurtamone, mesotrione, sulcotrione, isoxachlortole, isoxaflutole, benzofenap, pyrazolynate, pyrazoxyfen, benzobicyclon, amitrole, clomazone, aclonifen, 4-(3-trifluoromethylphenoxy)-2-(4-trifluoromethylphenyl)pyrimidine and 3-heterocyclyl-substituted benzoyl derivatives of the formula (cf. WO-A-96/26202, WO-A-97/41116, WO-A-97/41117 and WO-A-97/41118)

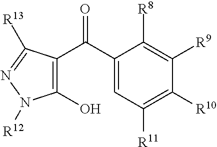

in which the substituents R⁸ to R¹³ are each defined as follows:
R₈, R₁₀ are hydrogen, halogen, C₁-C₅-alkyl, C₁-C₅-haloalkyl, C₁-C₅-alkoxy, haloalkoxy, C₁-C₅-alkylthio, C₁-C₅-alkylsulfinyl or C₁-C₅-alkylsulfonyl;
R⁹ is a heterocyclic radical from the group consisting of thiazol-2-yl, thiazol-4-yl, thiazol-5-yl, isoxazol-3-yl, isoxazol-4-yl, isoxazol-5-yl, 4,5-dihydroisoxazol-3-yl, 4,5-dihydroisoxazol-4-yl and 4,5-dihydroisoxazol-5-yl, where the radicals mentioned may bear one or more substituents; for example, they may be mono-, di-, tri- or tetra-substituted by halogen, C₁-C₄-alkyl, C₁-C₄-alkoxy, C₁-C₄-haloalkyl, C₁-C₄-haloalkoxy or C₁-C₄-alkylthio;
R¹¹=hydrogen, halogen or C₁-C₅-alkyl;
R¹²=C₁-C₆-alkyl;
R¹³=hydrogen or C₁-C₆-alkyl if the pH is <8;
mitosis inhibitors, such as benfluralin, butralin, dinitramine, ethalfluralin, fluchloralin, isopropalin, methalpropalin, nitralin, oryzalin, pendimethalin, prodiamine, profluralin, trifluralin, amiprofos-methyl, butamifos, dithiopyr, thiazopyr, propyzamide, chlorthal, carbetamide, chlorpropham and propham;

VLCFA inhibitors, such as acetochlor, alachlor, butachlor, butenachlor, delachlor, diethatyl, dimethachlor, dimethenamid, dimethenamid-P, metazachlor, metolachlor, S-metolachlor, pretilachlor, propisochlor, prynachlor, terbuchlor, thenylchlor, xylachlor, CDEA, epronaz, diphenamid, napropamide, naproanilide, pethoxamid, flufenacet, mefenacet, fentrazamide, anilofos, piperophos, cafenstrole, indanofan and tridiphane;
inhibitors of the biosynthesis of cellulose, such as dichlobenil, chlorthiamid, isoxaben and flupoxam;
herbicides, such as dinofenate, dinoprop, dinosam, dinoseb, dinoterb, DNOC, etinofen and medinoterb;
also: benzoylprop, flamprop, flamprop-M, bromobutide, chlorflurenol, cinmethylin, methyldymron, etobenzanid, pyributicarb, oxaziclomefone, triaziflam and methyl bromide.

Active ingredients used in crop protection can also be used to control pests (for example cockroaches, ants, termites inter alia) in an urban situation (for example residential developments, domestic and garden sectors, restaurants, car parks, hotel buildings, industrial areas inter alia) and are a further group of suitable effect substances specifically for these applications.

It is also possible to formulate active ingredients for controlling pests from the field of vertebrates (for example rats, mice inter alia) with the processes according to the invention, and to employ the resulting active ingredient formulations for corresponding pest control in agriculture and in an urban situation.

Additionally suitable are active ingredients for pharmaceutical use, especially those for oral administration. The process according to the invention is in principle applicable to a multitude of active ingredients irrespective of the medical indication.

Particular mention should be made of water-soluble active ingredients for pharmaceutical use, especially those for oral administration. This relates both to prescription-only and over the counter active ingredients. The invention is in principle applicable to a multitude of therapeutic, prophylactic or diagnostic active ingredients irrespective of the medical indication. Nonlimiting examples of usable active ingredient classes comprise anti-inflammatory agents, vasoactive agents, infection-inhibiting agents, anesthetic agents, growth-promoting agents. Compound classes usable in principle are proteins, peptides, nucleic acids, mono-, di-, oligo- and polysaccharides, proteoglycans, lipids, low molecular weight synthetic or natural organic active ingredients, or inorganic compounds or elements, for example silver.

Nonlimiting examples of suitable sparingly water-soluble active pharmaceutical ingredients are specified in the following table:

| Active | Imperial | Solubility in | |
|---|---|---|---|
| ingredient | formula | water [g/l] | |
| Felodipine | C₁₈H₁₉Cl₂NO₄ | 4.53E−03 | (22° C.) |
| Indomethacin | C₁₉H₁₆ClNO₄ | 1.4E−02 | (25° C.) |
| Piroxicam | C₁₅H₁₃N₃O₄S | 2.3E−02 | (RT) |
| Carbamazipine | C₁₅H₁₂N₂O | 9.451E−01 | (RT) |
| 17-β-Estradiol | C₁₈H₂₄O₂ | 1.836E−05 | (25° C.) |
| Clotrimazole | C₂₂H₁₇ClN₂ | <1.0E−02 | (25° C.) |
| Ketoconazole | C₂₆H₂₈Cl₂N₄O₄ | 8.0E−02 | (37° C.) |
| Cinnarizine | C₂₆H₂₈N₂ | 7.5E−01 | |
| Griseofulvin | C₁₇H₁₇ClO₆ | 3.685E−05 | (25° C.) |
| Ibuprofen | C₁₃H₁₈O₂ | 2.1E−02 | (25° C.) |

Examples of water-soluble active pharmaceutical ingredients are especially active cough-inducing and mucolytic ingredients, for example guaiacol glycol ether (also known as guaifenesin) and derivatives thereof.

Further preferred active pharmaceutical ingredients are antibodies and other proteins used in pharmacy, for example enzymes or peptides, or nucleic acids.

(vii) Active Ingredient Release from the Formulations

The active ingredients can be released from the formulations produced by the processes according to the invention by desorption into suitable solvents, by the degradation of the inventive sheetlike biopolymer structures (e.g. protein films, protein fibers, protein nonwovens) by proteases, or by dissolution of the inventive sheetlike biopolymer structures (e.g. protein films, protein fibers, protein nonwovens) by suitable solvents. Suitable solvents for the desorption are all solvents or solvent mixtures in which the active ingredient can be dissolved. Suitable proteases can be added in a controlled manner as technical proteases to a suspension of the inventive sheetlike biopolymer structures (e.g. protein films, protein fibers, protein nonwovens), or may occur naturally at the desired site of use of the effector molecules, for example proteases of the digestive tract, e.g. gastric or intestinal proteases, or proteases released by microorganisms. Solvents which can dissolve the inventive sheetlike biopolymer structures are, for example, fluorinated alcohols, for example hexafluoroisopropanol or trifluoroethanol, ionic liquids, for example EMIM acetate, aqueous solutions of chaotropic salts, for example urea, guanidinium hydrochloride and guanidinium thiocyanate, or organic acids, for example formic acid, and mixtures of these solvents with other organic solvents. The rate and the kinetics of the release of the effector molecules can be controlled, for example, by the loading density with active ingredients and the size of the inventive sheetlike biopolymer structures, or their ratio of volume to surface area.

The invention further provides for the use of the protein-containing sheetlike structures (e.g. protein films, protein fibers, protein nonwovens) produced utilizing the amphiphilic self-assembly proteins described for storage, for transport or for release of active ingredients in pharmaceutical products, cosmetic products, crop protection products, foods and animal feeds. The inventive sheetlike structures further serve to protect the packaged active ingredients from environmental influences, for example oxidative processes or UV radiation, or from destruction by reaction with other constituents of the products or from degradation by particular proteases. The active ingredient can be released from the protein-containing sheetlike structures by desorption, proteolytic degradation, controlled release or slow release, or a combination of these mechanisms.

The inventive protein-containing sheetlike structures (e.g. protein films, protein fibers, protein nonwovens) and active ingredients formulated therewith in pharmaceutical products are preferably to be taken perorally. This can increase the stability of the active ingredients as they pass through the stomach, since there is no proteolytic degradation of the inventive sheetlike structures under the conditions which exist therein. The active ingredients are then released in the intestine from the active ingredient-comprising sheetlike protein structures which have been taken perorally and may also be compressed to tablets or capsules. However, the active ingredients can be released under gastric conditions by desorption or diffusion.

In pharmaceutical products, foods and animal feeds or crop protection products, a formulation of active ingredients with the processes according to the invention using the biopolymers described, especially amphiphilic, self-assembly proteins, can also lead to an increased bioavailability of the active ingredients. The packaging of active pharmaceutical ingredients in sheetlike protein structures can also lead to improved absorption through the intestinal mucosa. Crop protection products can be protected from washout processes by encapsulation or embedding in sheetlike protein structures. Particular active ingredient particle sizes which are taken up or absorbed better or have better bioavailability can be established by packaging in sheetlike protein structures.

By varying the amino acid sequence of the amphiphilic self-assembly proteins described, or fusion with additional protein or peptide sequences, it is possible to generate structures which specifically recognize particular surfaces, for example skin, hair, leaves, roots or intestinal or vascular surfaces, or are recognized and bound by these surfaces or the receptors present.

It is thus possible to more effectively bring the active ingredients formulated with the amphiphilic self-assembly proteins described to the desired site of action, or to improve the active ingredient absorption. The bioavailability of active pharmaceutical ingredients or active ingredients in foods and animal feeds can be increased when they are packaged in sheetlike protein structures (e.g. protein films, protein fibers, protein nonwovens) which are additionally present fused to or associated with proteins which bind to particular surface markers (e.g. receptors) of cells of the gastrointestinal tract (e.g. mucosa cells). Such proteins are, for example, the MapA protein or the collagen-binding protein CnBP from Lactobacillus reuteri (Miyoshi et al., 2006, Biosci. Biotechnol. Biochem. 70:1622-1628), or functionally comparable proteins from other microorganisms, in particular the natural gastrointestinal flora. The binding proteins described mediate adhesion of the microorganisms to cell surfaces. Coupling or fusion of the binding proteins to the amphiphilic self-assembly proteins described would direct active ingredient-comprising sheetlike protein structures originating therefrom in a more controlled manner to appropriate absorption sites, or they would remain longer at these sites, which results in prolonged and improved active ingredient release and absorption.

In addition, it is possible by varying the amino acid sequence of the amphiphilic self-assembly proteins described for the active ingredient formulation, or fusion with additional protein or peptide sequences, to direct active ingredients in a controlled manner to desired sites of action, in order thus to achieve, for example, a higher specificity, lower consumption of active ingredient or active ingredient dose, or a faster or stronger effect.

It is additionally possible to add further substances to the spinning solution, in order, for example, to have a later influence on the crystallization of the active ingredient in the sheetlike structures (for example to inhibit it), or to achieve preferred use properties, such as altered bioavailability. Preferred additives are, for example, ionic (cationic or anionic) and nonionic surfactants. Suitable amounts of the additives in the spinning solution are 0.01% by weight to 5% by weight.

In addition, substances which enable disintegration of the tablets or capsules and hence improved dispersion of the sheetlike biopolymer structure compressed to the tablets or capsules can be added to the spinning solution or the sheetlike structures produced therefrom.

(viii) Fibrous Sheetlike Structures (Nonwovens) for Wound Treatment and Body Care

The inventive nonwovens can be combined with wound treatment products or body care products known per se, i.e. incorporated into them or applied to them. Conventional wound dressings, for example gauze or nonwoven or absorptive pads, are usually woven or nonwoven fabrics of cotton, viscose or synthetic fibers, such as polyamide, polyethylene or polypropylene. These can be impregnated with hydrophobic fatty ointments and exhibit a high absorptivity, which promotes the draining of excess wound exudate, tissue fragments and bacteria.

However, frequent changing of bandages is necessary, and drying-out of the wound is sometimes observed. This can lead to adhesion of the wound to dried wound secretion, or to the growth of young epithel tissue into the pad. Changing the bandage leads in this case to new lesions, which distinctly retards the healing process.

Modern wound dressings should therefore ensure an ideally moist wound environment. The materials used should be capable of forming gel to absorb large amounts of moisture, as is the case, for example, for polyacrylates and alginates, or hydrocolloidal products based on carboxymethyl cellulose. Due to the high absorption capacity thereof, these products are used primarily in the case of moderately to severely weeping wounds. In the case of drying out and in the case of necrotic wounds, these dressings can stick and, due to the great shrinkage, there is the risk that the wound will be traumatized again by tearing off the tissue below it.

An extensive range of wound materials and designs for control of wound healing have been described, but these are attuned very specifically to particular fields of use and substantially to clinical use. In general, what are called sandwich dressings are provided with the desired profile of properties; for instance, the first layer usually consists of a nonadhering layer (for example polyurethane-based foams or paraffin gauze) and of a second layer with a high absorption capacity for wound secretion, for example cellulose pads.

The inventive nonwovens constitute an inexpensive, easily fittable product which can be used as a healing-promoting textile separating layer from the wound, which permits the diffusion of oxygen and wound secretion due to the porosity thereof, but elastically seals the wound and is absorbed during healing.

The inventive materials can also be used in simpler wound care and permit the use of multilayer, costly dressings to be dispensed with.

Particular advantages of inventive fibrous sheetlike structures, such as biocompatibility, extensibility, nontoxicity, biodegradability (especially proteolytic degradability), good regulation of moisture content, make them suitable candidates for the production of products for treatment of chronic or nonchronic wounds and for body care.

Active ingredient-free or active ingredient-containing fibrous sheetlike structures produced in accordance with the invention are particularly suitable for production of wound care products and hygiene articles. In these cases, they can be used as such or applied to a suitable textile fabric or polymeric carrier material known per se.

For this purpose, it is possible to combine different materials in a manner known per se and to process them further to give multilayer products. According to the end use, it is possible to combine materials such as PE, PET or PU films and aluminum composite film, nonwovens, substrates, silicone papers, laminates, etc. with the inventive fibrous sheetlike structure.

In the case of production of the medical products comprising the inventive sheetlike biopolymer structures (for example wound dressings or plasters) or hygiene products (wipes, diapers, napkins, etc.), or in the case of use of the inventive biopolymer nonwovens in corresponding applications, the carrier substrate or the carrier material used for the sheetlike structure may be the medical or hygiene product to be coated itself, or parts or individual layers thereof.

The invention will now be illustrated in detail with reference to the nonlimiting examples which follow.

### EXPERIMENTAL SECTION

### General Section:

### a) Electrospinning Processes

The electrospinning apparatus suitable for performance of the process according to the invention comprises a syringe provided at its tip with a capillary nozzle connected to one pole of a voltage source, to accommodate the inventive formulation. Opposite the exit of the capillary nozzle is arranged, at a defined distance, a square counterelectrode connected to the other pole of the voltage source, which functions as the collector for the fibers formed.

A further possible apparatus for performance of the process according to the invention comprises a roller which rotates within a vessel containing spinning solution. The roller may be smooth or have physical structuring, for example needles or grooves. On each rotation of the roller, the spinning solution gets into the strong electrical field, and several material streams are formed. The counterelectrode is above the spinning electrode. The fibers are deposited on a carrier nonwoven, e.g. polypropylene.

For example, it is possible to use a Nanospider apparatus from Elmarco. The voltage is about 82 kV at an electrode distance of 18 cm. The temperature is about 23° C. and the relative air humidity 35%. A serrated electrode is used for spinning. In order to achieve a sheetlike protein structure of maximum thickness (e.g. protein films, protein fibers, protein nonwovens), the carrier nonwoven is left stationary. Alternatively, the carrier nonwoven can also be moved with an advanced rate to achieve relatively thin sheetlike protein structure layers in a defined manner. The sheetlike protein structures obtained from the batch (e.g. protein films, protein fibers, protein nonwovens) are subsequently dried at 40° C. under reduced pressure overnight. The layer thickness of the sheetlike protein structures is determined with the Millitron layer thickness measuring instrument (from Mahr Feinprüf, Germany).

### b) Active Ingredient Release Tests

The release of active ingredients from the sheetlike protein structures was tested in two different tests.

Active ingredient formulations to be taken perorally, for example guaiacol glyceryl ether and clotrimazole (pressed to tablets) were analyzed in synthetic gastric juice (0.1 g of NaCl; 0.16 g of pepsin; make up 0.35 ml of HCl to 50 ml, pH 1-2) and synthetic intestinal juice (dissolve 3.4 g of KH₂PO₄ in 12.5 ml of water+make up 3.85 ml of 0.2N NaOH to 25 ml+make up 0.5 g of pancreatin to 50 ml, pH 6.8), in order to simulate the release of active ingredient under proteolytically active conditions in the digestive tract. Control tests (without proteases) were effected in 5 mM potassium phosphate buffer (pH 8.0), and only a small release of active ingredient should be observed under these conditions. 20 ml of the particular digestive juice or buffer were added per tablet, and the mixtures were incubated with slight agitation at 37° C. and 80 rpm. At different times, 500 μl of sample in each case were taken for an active ingredient quantification by means of HPLC or a photometer. In order also to detect active ingredient aggregates formed after the release in the case of sparingly water-soluble active ingredients, for example clotrimazole, the absorption photometry quantification was performed after extraction with THF (3 ml of supernatant+3 ml of THF+spatula-tip of NaCl, vigorous vortexing, 1 min at 15 000×g, analyze upper phase, dilute if appropriate).

In the case of other active ingredients (active pharmaceutical ingredients not taken perorally or other active ingredients, for example active cosmetic ingredients or active crop protection ingredients), for example Uvinul A+ and metazachlor, the release analysis was effected by admixing defined amounts of sheetlike protein-active ingredient structures with unspecific proteinase K solution. The sheetlike protein-active ingredient structures were incubated in 0.25-0.5% [w/v] proteinase K (Roche, Germany; dissolved in 5 mM potassium phosphate buffer) with agitation at 120-150 rpm. At different times, the still-intact sheetlike protein-active ingredient structures were removed by centrifugation, the supernatants were admixed with a 4-5-fold excess of THF and the active ingredient content was subsequently determined by absorption photometry. In all experiments, the amounts of active ingredient released were determined after comparison with an active ingredient-specific calibration series.

### EXAMPLE 1

### Production of the C16 Spider Silk Protein

The C16 spider silk protein was produced by biotechnological means using plasmid-containing Escherichia coli expression strains. The design and cloning of the C16 spider silk protein (also known as ADF4) are described in Hümmerich et al. (Biochemistry 43, 2004, 13604-13012). In contrast to the process described therein, C16 spider silk protein was produced in E. coli strain BL21 Gold (DE3) (Stratagene). It was grown in Techfors fermenters (Infors HAT, Switzerland) using a minimal medium and fed-batch techniques.

Minimal medium: 2.5 g/l citric acid monohydrate
4 g/l glycerol
12.5 g/l potassium dihydrogenphosphate
6.25 g/l ammonium sulfate
1.88 g/l magnesium sulfate heptahydrate
0.13 g/l calcium chloride dihydrate
15.5 ml/l trace element solution (40 g/l citric acid monohydrate;
11 g/l zinc(II) sulfate heptahydrate; 8.5 g/l diammonium iron(II) sulfate heptahydrate; 3 g/l manganese(II) sulfate monohydrate;
0.8 g/l copper(II) sulfate pentahydrate; 0.25 g/l cobalt(II) sulfate heptahydrate)
3 ml/l vitamin solution (6.3 mg/ml thiamine hydrochloride;
0.67 mg/ml vitamin B12)
pH 6.3
   Feed solution: 790 g/l glycerol
6.9 g/l citric acid monohydrate
13.6 g/l sodium sulfate
1.05 g/l diammonium iron(II) sulfate heptahydrate
13 mg/l thiamine hydrochloride

The cells were grown at 37° C. up to an OD₆₀₀ of 100, which was followed by the induction of protein expression with 0.1 mM isopropyl β-D-1-thiogalactopyranoside (IPTG). At the end of fermentation (8 to 12 hours after induction), the cultures were harvested. The main proportion of the protein was present in “inclusion bodies”.

After cell harvesting, the pellet was resuspended in 20 mM 3-(N-morpholino)propanesulfonic acid (MOPS), pH 7.0 (5 l of buffer per kilogram of wet material). This was followed by cell disruption using a Microfluidizer M-110EH (Microfluidics, US) at pressures of 1200 to 1300 bar. After sedimentation, the pellet after disruption comprised, as well as the inclusion bodies, also cell fragments and membrane constituents, which were removed by two wash steps. In a first wash step, the pellet was resuspended in 2.5 volumes of Tris buffer (50 mM Tris/HCl, 0.1% Triton X-100, pH 8.0) and then the remaining solids were sedimented by centrifugation. A second wash step was effected using Tris buffer (50 mM Tris/HCl, 5 mM EDTA, pH 8.0). The pellet obtained once again after sedimentation was virtually free of membrane and cell fragments.

The cleaned inclusion bodies were dissolved in guanidinium thiocyanate (Roth, Germany), with addition of 1.6 g of guanidinium thiocyanate per 1 g of pellet (wet mass). The inclusion bodies dissolved while stirring with gentle heating (50° C.). To remove any insoluble constituents present, a centrifugation was subsequently carried out. In order to obtain an aqueous C16 spider silk protein solution, a 16-hour dialysis was then carried out against 5 mM potassium phosphate buffer (pH 8.0) (dilution factor of the dialysis: 200).

Contaminating E. coli proteins formed aggregates in the dialysis, which were removable by centrifugation. The protein solution obtained had a purity of ˜95% C16 spider silk protein.

The resulting aqueous protein solution can either be used directly for electrospinning or, for the purpose of better storability, processed further to protein microbeads. To produce C16 protein microbeads, the aqueous C16 spider silk protein solution was admixed with 0.25 volume of a 4 molar ammonium sulfate solution. Under the action of the ammonium sulfate, the protein molecules assemble to form spherical structures, which are referred to here as microbeads. The microbeads were removed by centrifugation, washed three times with distilled water and then freeze-dried.

### EXAMPLE 2

### Formulation of Guaiacol Glyceryl Ether as an Effect Substance by Means of Electrospinning

In order to demonstrate the usability of the process described for the formulation of pharmaceutically active substances, especially those for treatment of coughs and respiratory disorders, by way of example, the active ingredient guaiacol glyceryl ether (also known as guaifenesin) was encapsulated by means of electrospinning in sheetlike C16 spider silk protein structures (e.g. protein films, protein fibers, protein nonwovens).

For the production of a spinnable solution, C16 spider silk protein microbeads (14% [w/w]) and the active ingredient guaiacol glyceryl ether (10% [w/w]) were dissolved together in formic acid (98-100% p.a.). A beaker was initially charged with 200 ml of formic acid, and then 50.4 g of C16 spider silk protein and 36 g of guaiacol glyceryl ether (from Sigma, Germany) were stirred in gradually. Once the substances had dissolved completely, the solution was made up to 360 g with formic acid (98-100%).

Alternatively, it is also possible to use aqueous C16 spider silk protein solution (see example 1) as the starting material basis. The active ingredient is then dissolved directly in the aqueous protein solution or, in the case of use of relatively high active ingredient concentrations, predissolved in an alternative solvent (e.g. formic acid) and then mixed with the protein solution. In order to increase the viscosity of the spinning solution, it is then additionally possible to add water-soluble polymers or aqueous polymer dispersions.

The solution of C16 spider silk protein and guaiacol glyceryl ether was spun as described above in an Elmarco Nanospider apparatus for 3 hours. The layer thickness of the resulting sheetlike protein structures was determined with the Millitron layer thickness measuring instrument (from Mahr Feinprüf, Germany), and was 0.01 - 0.2 mm.

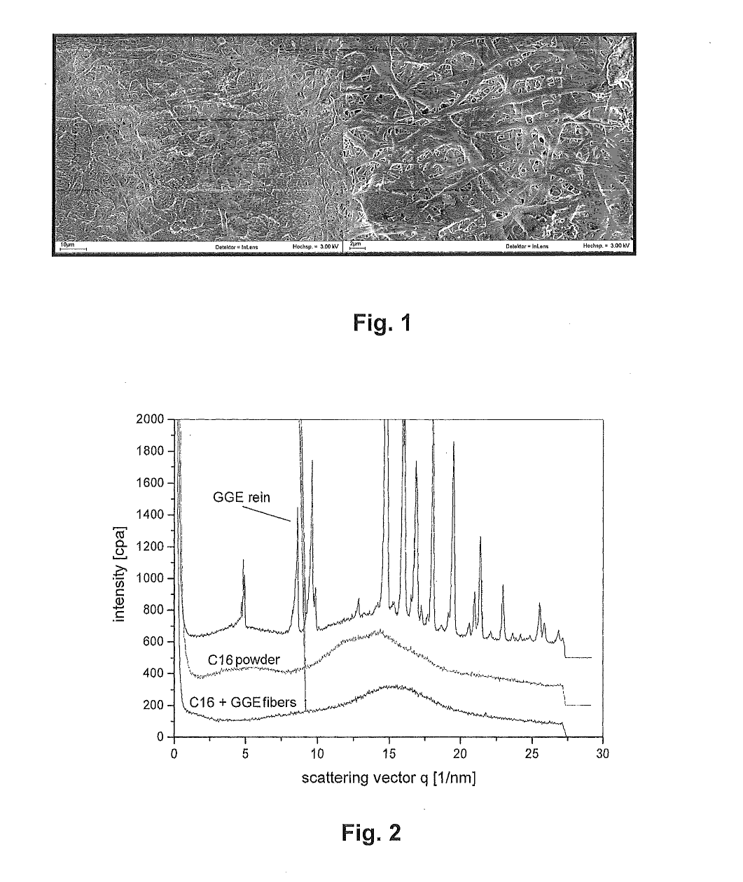

The electron microscope analysis of the thus produced sheetlike C16 spider silk protein structures with incorporated guaiacol glyceryl ether showed that the structures are principally fibers having a diameter up to 2 μm and lower (FIG. 1).

In contrast to pure guaiacol glyceryl ether, x-ray diffraction does not show any crystalline peaks in the C16 spider silk protein/guaiacol glyceryl ether formulation (FIG. 2). Accordingly, it can be assumed that the active ingredient has been encapsulated in amorphous form or as a solid solution, which can positively influence the bioavailability thereof.

In order to test active ingredient release from a very relevant administration form, the sheetlike C16 spider silk protein structures were used to press tablets. In each case 300 mg of material were pressed under reduced pressure and at pressure 100 bar in a KBr press (from Paul-Otto-Weber, Germany) for approx. 10 min. The tablets had a diameter of about 13 mm and a thickness of about 2 mm.

The release of guaiacol glyceryl ether from the tablets was determined, after treatment with synthetic gastric juice and synthetic intestinal juice, by means of HPLC (column: Luna C8(2), 150*3.0 mm [from Phenomenex, Germany]; precolumn: C18 ODS; detection: UV 210nm; eluent A: 10 mM KH₂PO₄, pH 2.5; eluent B: acetonitrile). While only a maximum of 20% of the encapsulated amount of active ingredient is released in the control experiments (buffer), 100% release is achieved in gastric and intestinal juice within 24 h, controlled by the enzymatic activities present (proteases) (FIG. 3). Both in gastric juice and in intestinal juice, the guaiacol glyceryl ether active ingredient is released continuously. About 65% of the active ingredient is released in the first 8 h in the experiments with intestinal juice, whereas about 80% of the active ingredient is already released within this time in the gastric juice (FIG. 3).

In order to determine the proportion of guaiacol glyceryl ether yet to be released from the formulation after 24 h, the mixtures containing the remaining C16 spider silk protein aggregates were adjusted to pH 7.0, in each case 100 μl of proteinase K (435 U/ml, Roche, Germany) were added, and the mixtures were incubated further at 37° C. and 120 rpm until all aggregates had dissolved completely. Subsequently, the active ingredient content in solution was quantified by means of HPLC analysis. As a result, it was possible to use the end value and the intermediate values determined beforehand to determine the loading density of the C16 spider silk protein formulation with the guaiacol glyceryl ether active ingredient. The loading density for all tablets examined was between 31 and 33% [w/w], which gave an average loading density of the sheetlike C16 spider silk protein structure pressed to tablets with 32.2% [w/w] guaiacol glyceryl ether (tab. 1).

TABLE 1
Loading densities of the C16 spider silk protein formulation
(tablets) with the guaiacol glyceryl ether active ingredient.

| | Tablet | GGE in | mg of GGE | Loading |
|---|---|---|---|---|
| | mass | solution | per mg | density |
| Experiment | [mg] | [mg] | of tablet | [%] |
| Buffer | 300 | 97.4 | 0.325 | 32.5 |
| Gastric juice | 299 | 94.7 | 0.317 | 31.7 |
| Intestinal juice | 300 | 97.4 | 0.325 | 32.5 |

| | |
|---|---|
| Average loading density | 32.2 |

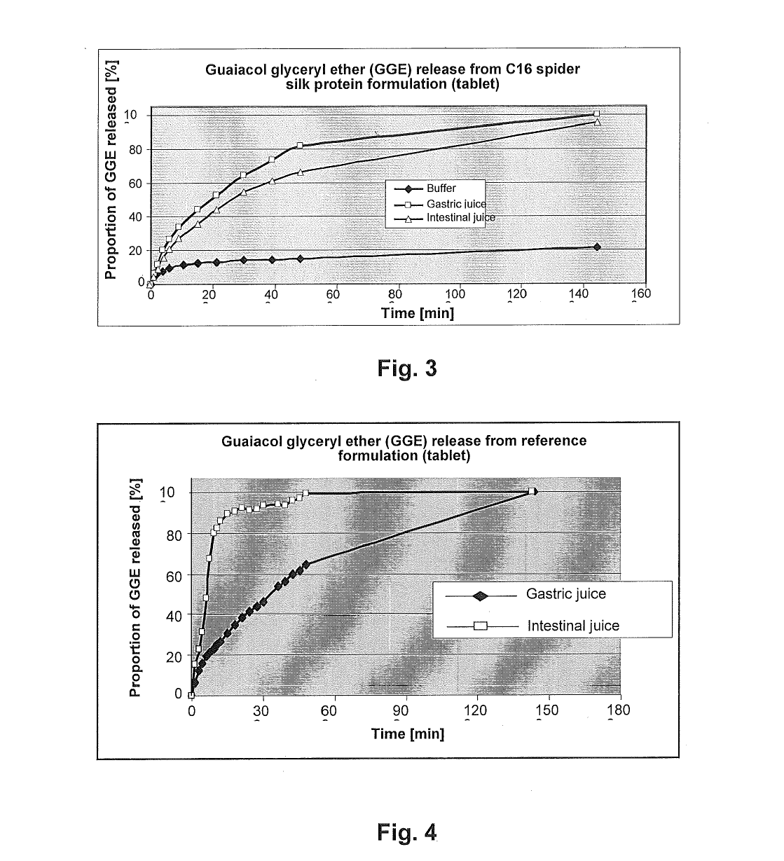

Control experiments with a formulated reference sample of the guaiacol glyceryl ether active ingredient (tablets of the Mucinex® brand, from Adams Respiratory Therapeutics) show a continuous, delayed active ingredient release only under gastric conditions (FIG. 4). Given an average gastric residence time of the active ingredient formulation of 2-5 hours, a maximum of 50% active ingredient would be released at this time. Under intestinal conditions, about 90% of the active ingredient is released from the Mucinex® formulation within a short time (2-3 hours) (FIG. 4).

On the basis of the test results shown in FIG. 4, it can be concluded that a continuous, delayed guaiacol glyceryl ether release and hence also the absorption thereof in the case of Mucinex® tablets does not take place under gastric conditions, and a majority of the active ingredient is thus lost via excretion processes. Inventive C16 spider silk protein formulations of the guaiacol glyceryl ether active ingredient, in contrast, exhibit continuous, delayed release under gastric conditions and also under intestinal conditions, which would promote longer-lasting active ingredient absorption. Accordingly, formulations of the guaiacol glyceryl ether active ingredient with amphiphilic self-assembly proteins would be usable much more universally, and still allow, even after passage through the stomach, continuous, delayed active ingredient release and subsequently active ingredient absorption.

### EXAMPLE 3

### Formulation of Clotrimazole as an Effect Substance by Means of Electrospinning

In order to show the usability of the process described for the formulation of further pharmaceutically active, especially sparingly water-soluble, substances, the active ingredient clotrimazole, by way of example, was encapsulated by means of electrospinning in sheetlike C16 spider silk protein structures (e.g. protein films, protein fibers, protein nonwovens).

For the production of a spinnable solution, C16 spider silk protein microbeads (14% [w/w]) and the active ingredient clotrimazole (10% [w/w]) were dissolved together in formic acid (98-100% p.a.). A beaker was initially charged with 200 ml of formic acid, and then 50.4 g of C16 spider silk protein and 36 g of clotrimazole (from Sigma, Germany) were stirred in gradually. Once the substances had dissolved completely, the solution was made up to 360 g with formic acid.

Alternatively, it is also possible to use water-soluble C16 spider silk protein solution (see example 1) as the starting material basis. The active ingredient is then dissolved directly in the aqueous protein solution or, in the case of use of relatively high active ingredient concentrations, predissolved in an alternative solvent (e.g. formic acid) and then mixed with the protein solution. In order to increase the viscosity of the spinning solution, it is then additionally possible to add water-soluble polymers or polymer dispersions.

The solution of C16 spider silk protein and clotrimazole was spun as described above in an Elmarco Nanospider apparatus for 3 hours.

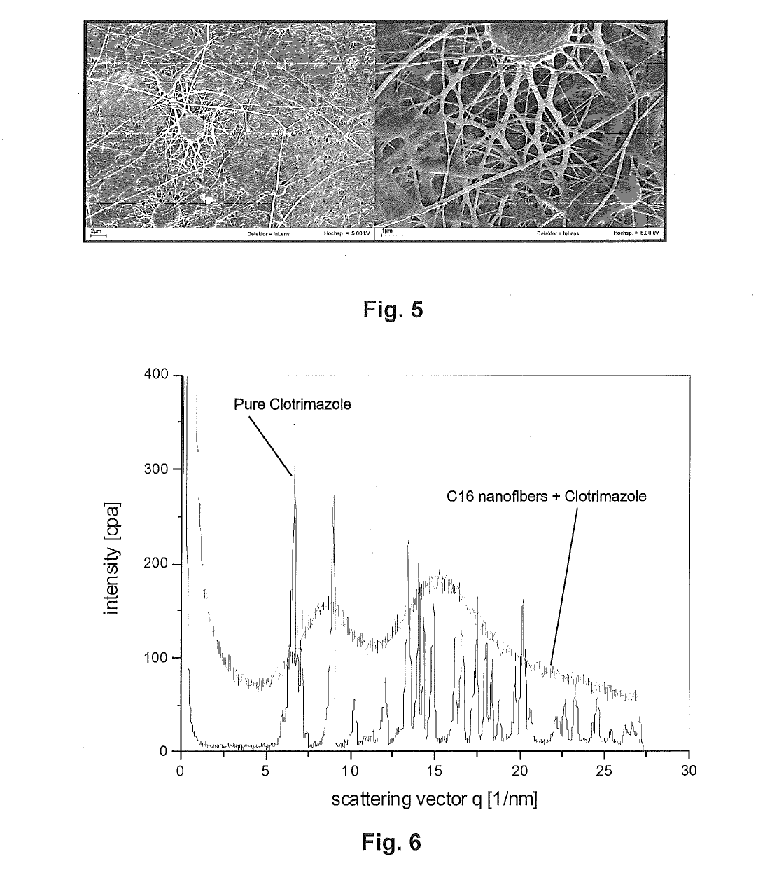

The electron microscope analysis of the thus produced sheetlike C16 spider silk protein structures with incorporated clotrimazole showed that the structures are principally fibers having a diameter about 50 nm up to 1 μm (FIG. 5).

In contrast to pure clotrimazole, x-ray diffraction does not show any crystalline peaks in the C16 spider silk protein/clotrimazole formulation (FIG. 6). Accordingly, it can be assumed that the active ingredient has been encapsulated in amorphous form or as a solid solution, which can positively influence the bioavailability thereof.

As already described in example 2, the sheetlike C16 spider silk protein structures comprising encapsulated clotrimazole active ingredient were also used to press tablets. In order to determine the release kinetics of the active ingredient, as described in example 2, the tablets were incubated in synthetic gastric juice, synthetic intestinal juice and 5 mM potassium phosphate buffer (control). The clotrimazole released was quantified on the basis of its poor water solubility (and hence tendency to form aggregates in aqueous systems) after extraction of the supernatant with THF by absorption photometry determination at 262 nm.

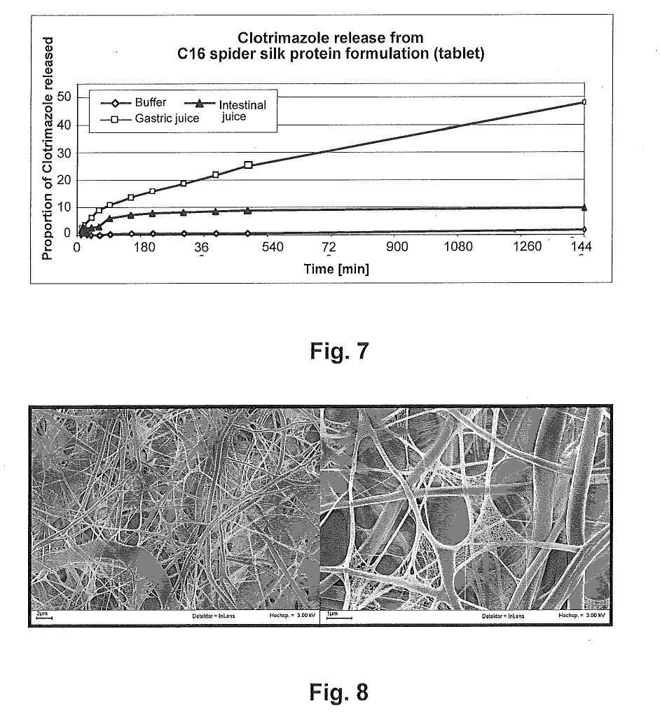

While only a maximum of 2% of the amount of active ingredient encapsulated is released in the control experiment (buffer without proteases), about 50% release is achieved within 24 h in gastric juice, controlled by the enzymatic activity present (proteases) (FIG. 7). In the course of this, the clotrimazole active ingredient is released continuously. In intestinal juice, in contrast, only about 20% of the active ingredient is released after 24 h (FIG. 7). The C16 spider silk protein/clotrimazole formulation appears to be so stable at the comparatively neutral pH values which exist therein over the time range in question that only attenuated release is observed.

In order to determine the proportion of clotrimazole as yet unreleased from the formulation after 24 h, the mixtures comprising the proteolytically undegraded sheetlike C16 spider silk protein structures were admixed with 3 ml of THF and incubated with shaking for a further max. 48 h. Subsequently, the active ingredient content was quantified by absorption photometry at 262 nm. It was thus possible to use the end value and the previously determined intermediate values to determine the loading density of the C16 spider silk protein formulation with the clotrimazole active ingredient. The loading density for all tablets examined was between 27 and 33% [w/w], which gave an average loading density of the sheetlike C16 spider silk protein structure pressed to tablets with about 30% [w/w] clotrimazole (tab. 2).

TABLE 2
Loading densities of the C16 spider silk protein formulation
(tablets) with the clotrimazole active ingredient.

| | | | mg of | |
|---|---|---|---|---|
| | Tablet | Clotrimazole | clotrimazole | Loading |
| | mass | in solution | per mg | density |
| Experiment | [mg] | [mg] | of tablet | [%] |
| Buffer | 304 | 92.2 | 0.303 | 30.3 |
| Gastric juice | 302 | 99.1 | 0.328 | 32.8 |
| Intestinal juice | 299 | 82.0 | 0.274 | 27.4 |

| | |
|---|---|
| Average loading density | 30.2 |

### EXAMPLE 4

### Formulation of Metazachlor as an Effect Substance by Means of Electrospinning

In order to show the usability of the process described for the formulation of active crop protection ingredients, the active ingredient metazachlor, by way of example, was encapsulated by means of electrospinning in sheetlike C16 spider silk protein structures (e.g. protein films, protein fibers, protein nonwovens).

For the production of a spinnable solution, C16 spider silk protein microbeads (14% [w/w]) and the active ingredient metazachlor (10% [w/w]) were dissolved together in formic acid (98-100% p.a.). A beaker was initially charged with 200 ml of formic acid, and then 50.4 g of C16 spider silk protein and 36 g of metazachlor were stirred in gradually. Once the substances had dissolved completely, the solution was made up to 360 g with formic acid (98-100%).

Alternatively, it is also possible to use aqueous C16 spider silk protein solution (see example 1) as the starting material basis. The active ingredient is then dissolved directly in the aqueous protein solution or, in the case of use of relatively high active ingredient concentrations, predissolved in an alternative solvent (e.g. formic acid) and then mixed with the protein solution. In order to increase the viscosity of the spinning solution, it is then additionally possible to add water-soluble polymers or polymer dispersions.

The solution of C16 spider silk protein and metazachlor was spun as described above in an Elmarco Nanospider apparatus for 3 hours.

The electron microscope analysis of the thus produced sheetlike C16 spider silk protein structures with incorporated metazachlor showed that the structures are principally fibers having a diameter about 50 nm up to 500 nm (FIG. 8).

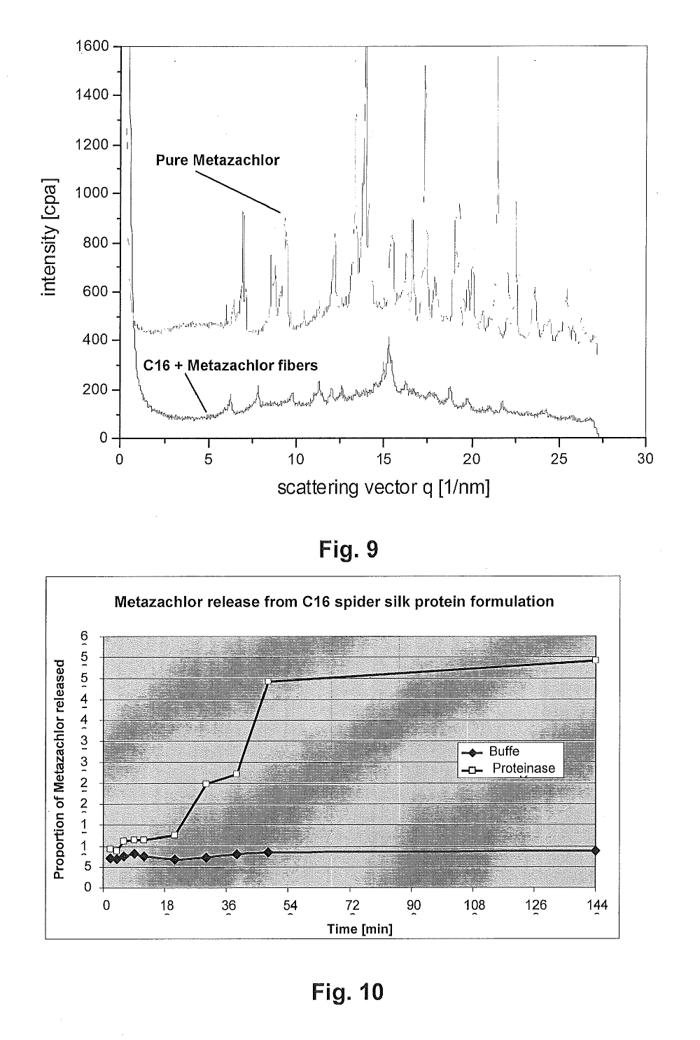

In x-ray diffraction, pure metazachlor exhibits significant crystalline proportions (FIG. 9). In contrast, the C16 spider silk protein/metazachlor formulation in x-ray diffraction has less marked semicrystalline regions which are attributable to the metazachlor active ingredient (FIG. 9).

To determine the loading density with the metazachlor active ingredient in two mixtures (1st mixture: 25 mg; 2nd mixture: 26 mg), the sheetlike spider silk protein structure produced was admixed with 2 ml of THF in each case and incubated at 1800 rpm with agitation for 5 h. The metazachlor active ingredient leached out quantitatively by the THF treatment was subsequently determined by absorption photometry at 264 nm. It was found that there was a loading density of about 40% [w/w] in mixture 1, and of about 45% [w/w] in the second mixture.

In order to determine the release kinetics of the active ingredient, the sheetlike C16 spider silk protein structures comprising encapsulated metazachlor were incubated in 5 mM potassium phosphate buffer admixed with 0.5% [w/v] proteinase K. The metazachlor released was quantified after removal of the still-intact sheetlike C16 spider silk protein structure by extraction of the supernatant with THF and subsequent absorption photometry determination at 264 nm.

While only about 10% of the amount of active ingredient encapsulated had been released after 24 h in the control experiment (buffer without proteinase K), it was possible to achieve the release of about 55% metazachlor within the same period in the proteinase K-containing experiment (FIG. 10). After 7 days, it was possible to achieve about 70% continuous active ingredient release from such a C16 spider silk protein/metazachlor formulation (not shown).

### EXAMPLE 5

### Formulation of Uvinul A+ as an Effect Substance by Means of Electrospinning

In order to show the usability of the process described for the formulation of active cosmetic ingredients, the active ingredient Uvinul A+, by way of example, was encapsulated by means of electrospinning in sheetlike C16 spider silk protein structures (e.g. protein films, protein fibers, protein nonwovens).

For the production of a spinnable solution, C16 spider silk protein microbeads (14% [w/w]) and the active ingredient Uvinul A+ (10% [w/w]) were dissolved together in formic acid (98-100% p.a.). A beaker was initially charged with 200 ml of formic acid, and then 50.4 g of C16 spider silk protein and 36 g of Uvinul A+ were stirred in gradually. Once the substances had dissolved completely, the solution was made up to 360 g with formic acid (98-100%).

Alternatively, it is also possible to use aqueous C16 spider silk protein solution (see example 1) as the starting material basis. The active ingredient is then dissolved directly in the aqueous protein solution or, in the case of use of relatively high active ingredient concentrations, predissolved in an alternative solvent (e.g. formic acid) and then mixed with the protein solution. In order to increase the viscosity of the spinning solution, it is then additionally possible to add water-soluble polymers or polymer dispersions.

The solution of C16 spider silk protein and Uvinul A+ was spun as described above in an Elmarco Nanospider apparatus for 3 hours.

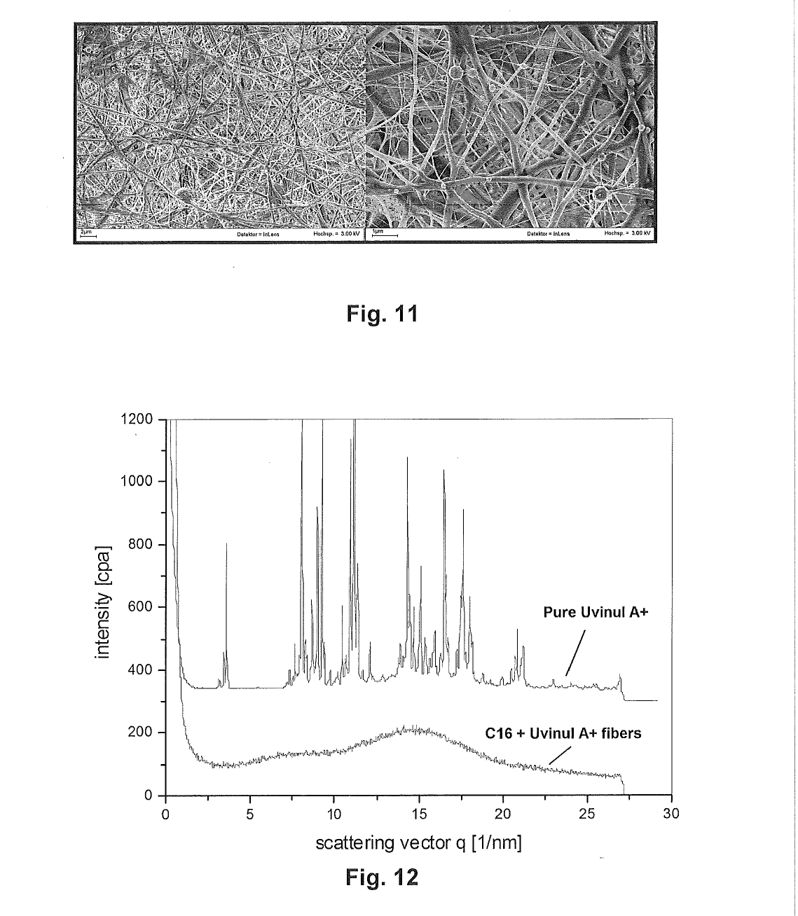

The electron microscope analysis of the thus produced sheetlike C16 spider silk protein structures with incorporated Uvinul A+ showed that the structures are principally fibers having a diameter about 50 nm up to 400 nm (FIG. 11).

In contrast to pure Uvinul A+, there are no crystalline peaks in the x-ray diffraction in the C16 spider silk protein/Uvinul A+ formulation (FIG. 12). Accordingly, it can be assumed that the active ingredient has been encapsulated in amorphous form or as a solid solution, which can positively influence the bioavailability thereof.

To determine the loading density with the active ingredient in two mixtures (1st mixture: 7.9 mg; 2nd mixture: 7.8 mg), the sheetlike C16 spider silk protein structure produced was admixed with 2 ml of THF in each case and incubated at 1800 rpm with agitation for 5 h. The Uvinul A+ active ingredient leached out quantitatively by the THF treatment was subsequently determined by absorption photometry at 352 nm. It was found that a loading density of about 25% [w/w] was present in mixture 1, and of about 26.2% [w/w] in the second mixture.

In order to determine the release kinetics of the active ingredient, the sheetlike C16 spider silk protein/Uvinul A+ structures were incubated in 5 mM potassium phosphate buffer admixed with 0.25% [w/v] proteinase K. The Uvinul A+ released was quantified, after removal of the still-intact sheetlike C16 spider silk protein structures, by extraction of the supernatant with THF and subsequent absorption photometry determination at 352 nm.

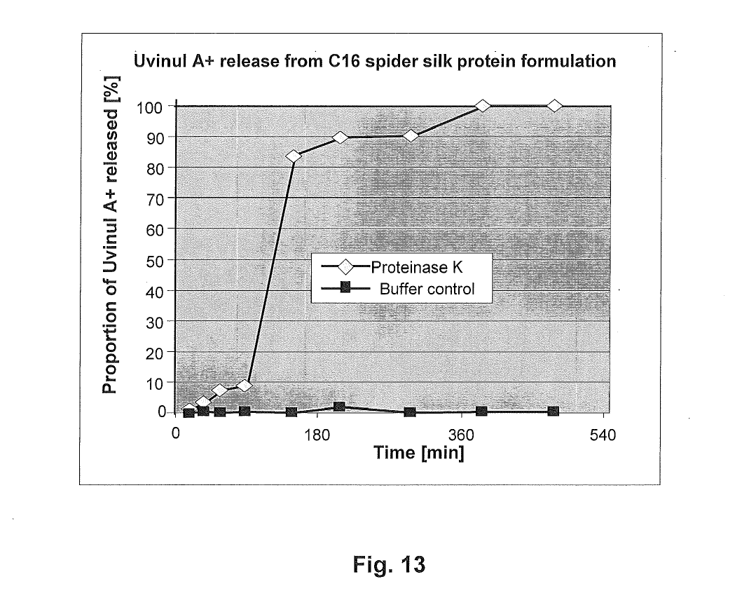

While no active ingredient was released in the control experiment (buffer without proteinase K) even after 24 h, the release of 100% Uvinul A+ was achieved after 6-7 hours in the proteinase K-containing experiment (FIG. 13).

### EXAMPLE 6

### Production of Fibers from Pure R16 and S16 Proteins by Means of Electrospinning

For the production of spinnable R16 or S16 protein solutions, R16 or S16 protein microbeads were used. These can be produced as described in WO 2008/155304.

Alternatively, a preparation can be effected as described in example 1. Plasmid vectors or E. coli production strains were used, which comprised coding DNA sequences for the R16 or S16 protein.

For the production of a spinnable solution, R16 protein microbeads were dissolved in an 18% [w/w] solution in formic acid (98-100% p.a.). The R16 protein was spun in a small batch to detect fiber formation. For this purpose, 0.36 g of R16 protein microbeads was dissolved in 1.64 g of formic acid and this was used to fill the syringe of the spinning system.

The R16 protein solution was spun with the aid of the nozzle-based electrospinning system. For this purpose, the protein solution was extruded in an electrical field under low pressure through a cannula connected to one pole of a voltage source. The electrostatic charge of the protein solution which resulted from the electrical field gave rise to a material flow directed at the counterelectrode, which solidified on the way to the counterelectrode and was deposited in the form of thin fibers on a glass microscope slide.

The following parameters were used:
Rel. air humidity 27%,
Spinning temperature 23° C.,
Electric voltage 60 kV,
Electrode distance 15 cm,
Cannula diameter 0.9 mm,
manual advance

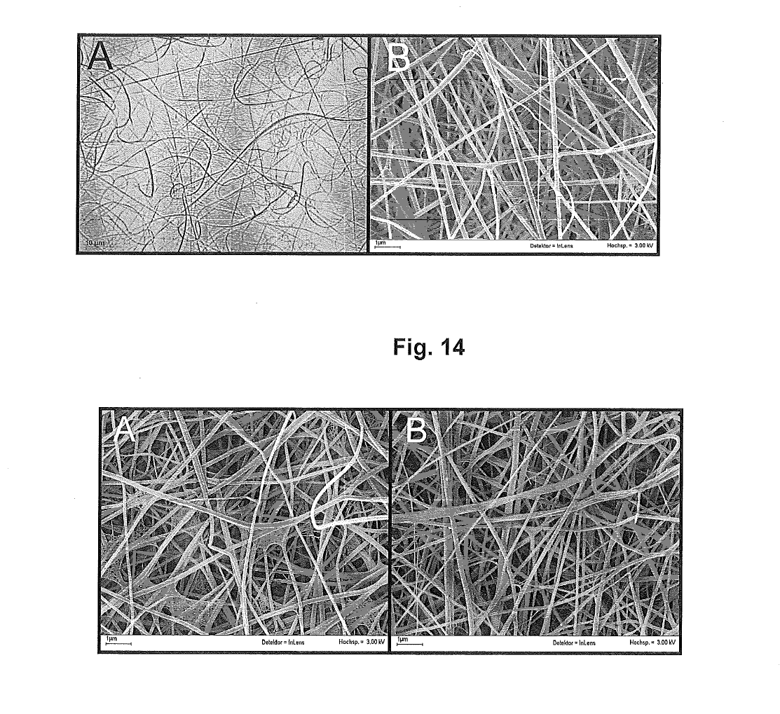

The electron microscope analysis of the sheetlike R16 protein structures thus produced showed that the solution was fiber-forming, and that the fibers were principally those having a diameter of about 200 nm up to 500 nm (FIG. 14A).

The S16 protein solution was spun with the Elmarco Nanospider apparatus. The solution used was present in a vessel in which a spinning electrode (roller) rotated permanently. In this case, the spinning electrode was an electrode based on metal wires. A portion of the formulation was constantly present on the surface of the wires. The electrical field between the roller and the counterelectrode (above the roller) at first caused formation of liquid jets from the formulation, which then lose solvent present and solidify on the way to the counterelectrode. The desired nanofiber web (textile sheetlike structure) formed on a polypropylene substrate which moved along between the two electrodes.

Microbeads of the S16 protein were dissolved in a 12% solution [w/w] in formic acid (98-100% p.a.). For the S16 mixture, a beaker was initially charged with 200 ml in each case of formic acid and then 40 g of S16 protein were stirred in gradually. Once the S16 protein had dissolved completely, the solution was made up to 340 g with formic acid (98-100%).

The following parameters were used:
Temperature: 24° C.
rel. air humidity: 22%
Voltage: 70-82 kV
Electrode distance: 25 cm
Spinning time: 1.5 h

The sheetlike S16 protein structure had fibers with a diameter of about 100 nm up to 300 nm (FIG. 14B).

In the case of production of medical products (e.g. wound dressings or plasters) or hygiene products (wipes, diapers, napkins, etc.) comprising sheetlike R16 or S16 protein structures, or the use of R16 or S16 protein nonwovens in corresponding applications, the carrier substrate or carrier substance used for the sheetlike structure may be the medical or hygiene product to be coated itself, or parts or individual layers thereof.

Alternatively, it is also possible to use aqueous R16 or S16 protein solutions (analogously to C16 spider silk protein, for production see example 1) as the starting material for the production of fibers/fibrous sheetlike structures. In order to increase the viscosity of the spinning solution or to obtain the viscoelasticity of the solutions, it is then additionally possible to add water-soluble polymers, polymer dispersions or biopolymers (e.g. proteins).

### EXAMPLE 7

### Formulation of Uvinul A+ as an Effect Substance in R16 and S16 Protein Nonwovens by Means of Electrospinning

In order to show the usability of the process described for the formulation of active ingredients, the active ingredient Uvinul A+, by way of example, was encapsulated by means of electrospinning in sheetlike R16 or S16 protein structures (e.g. protein films, protein fibers, protein nonwovens).

For the production of a spinnable solution, R16 protein microbeads (18% [w/w]) or S16 protein microbeads (12% [w/w]) and the active ingredient Uvinul A+ (10% [w/w]) were dissolved together in formic acid (98-100% p.a.). A beaker was initially charged with 200 ml of formic acid, and then 61.2 g of R16 protein or 40.0 g of S16 protein and 34 g of Uvinul A+ were stirred in gradually. Once the substances had dissolved completely, the solution was made up to 340 g with formic acid (98-100%).

Here too, it is alternatively possible to use aqueous R16 or S16 protein solutions (analogously to C16 spider silk protein, for production see example 1) as the starting material basis. The active ingredient is then dissolved directly in the aqueous protein solution or, in the case of use of relatively high active ingredient concentrations, predissolved in an alternative solvent (e.g. formic acid or THF) and then mixed with the protein solution. In order to increase the viscosity of the spinning solution, it is then additionally possible to add water-soluble polymers, polymer dispersions or biopolymers (e.g. proteins).

The solution of R16 protein or S16 protein and Uvinul A+ spun with the following parameters in the roller-based Elmarco Nanospider apparatus:

| | R16 protein/Uvinul A+ | S16 protein/Uvinul A+ |
|---|---|---|
| Voltage [kV] | 82 | 82 |
| Temperature [° C.] | 24 | 24.5 |
| Rel. air humidity [%] | 33 | 27.5 |
| Electrode separation [cm] | 25 | 25 |
| Spinning time [h] | 1 | 3 |

The sheetlike protein structures comprising incorporated Uvinul A+ thus produced had comparable fiber diameters to the experiments without active ingredient (FIG. 15).

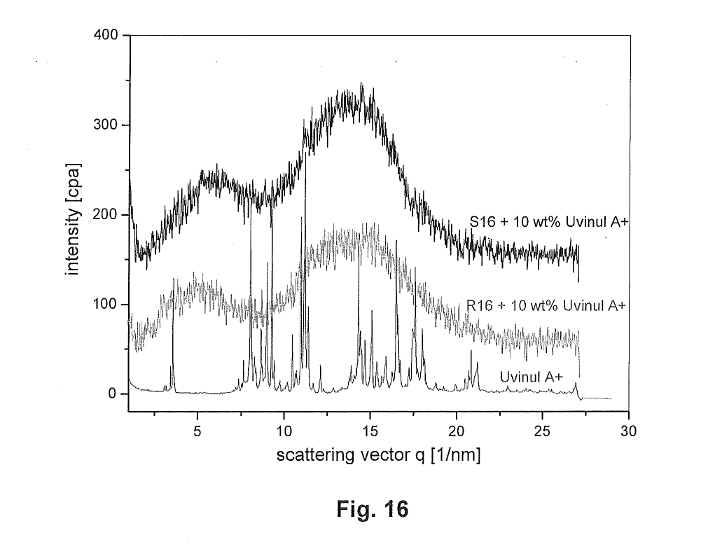

In contrast to pure Uvinul A+, the x-ray diffraction spectra did not show any crystalline peaks in the R16 protein/Uvinul A+ formulation (FIG. 16). Accordingly, it can be assumed that the active ingredient has been integrated in amorphous form into the fibers. In the S16 protein comprising Uvinul A+, it was possible to detect very weak crystalline signals. This suggests that the effect substance is present in semicrystalline form.

In a departure from the procedure detailed above, the release kinetics of the active ingredient from sheetlike R16 or S16 protein/Uvinul A+ structures were determined as follows. In each case 10 mg of the sheetlike R16 or 5 mg of the S16 protein/Uvinul A+ structures were incubated in 5 mM potassium phosphate buffer with 0.25% [w/v] proteinase K. For each planned sampling time, a mixture was made up in each case. The mixtures were incubated at 37° C. and 400 rpm in a Thermomixer (from Eppendorf). The Uvinul A+ released was quantified at the particular times, after removal of the still-intact sheetlike R16 or S16 protein structures, by extracting the supernatant with THF and subsequent absorption photometry determination at 352 nm.

To determine the loading density with the active ingredient, all samples made up for the release kinetics were extracted quantitatively with THF. In the samples in which there was incomplete degradation of the sheetlike protein structures, the sheetlike protein structure removed was also extracted with THF and then Uvinul A+ was quantified by absorption photometry. The two values (supernatant and pellet) were added to determine the total loading density. The loading densities at all times were subsequently used to determine the mean loading density. It was found that a Uvinul A+ loading density of about 33.5% [w/w] was present in the sheetlike R16 protein structure, and of about 49.6% [w/w] in the sheetlike S16 protein structure.

After 24 h, only 7.5% Uvinul A+ had been released from the sheetlike R16 structure in the control experiment (buffer without proteinase K). Within this period, 9.5% active ingredient was released from the sheetlike S16 structure in the control experiment. In both experiments, however, the sheetlike protein structures remained intact. Within the same period, the sheetlike S16 structure in the experiment with added proteinase K had degraded completely. The proteinase K-controlled degradation of the sheetlike R16 structure was much slower in comparison. After 24 h, isolated residues of the sheetlike R16 structure were still evident here in the mixture.

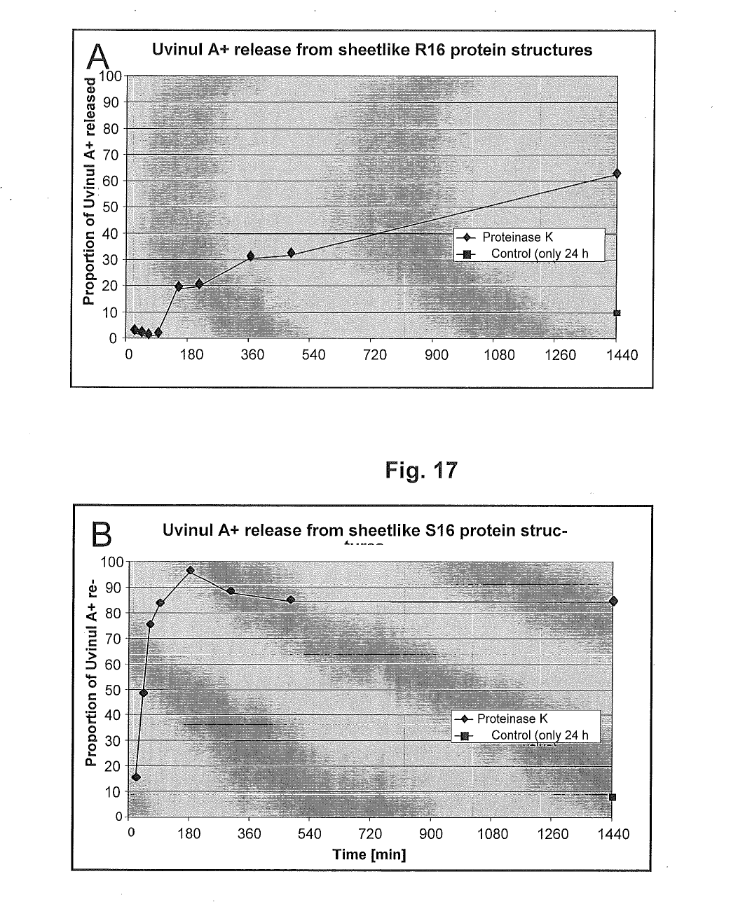

In the proteinase K-comprising R16 protein mixture, about 63% [w/w] of the Uvinul A+ had been released after 24 h (FIG. 17). In the S16 protein mixture, in contrast, all of the Uvinul A+ active ingredient had been released after only about 3 hours (FIG. 17).

Reference is made explicitly to the disclosure of the publications cited herein.

1. -37. (canceled)

38. An active ingredient-containing a fibrous sheetlike structure comprising a fibrous, polymeric, soluble and/or degradable active ingredient carrier and at least one active ingredient which is associated with the carrier and can be released by the fibrous sheetlike structure,
wherein the carrier comprises, as a polymer component, at least one biopolymer which may additionally have been chemically and/or enzymatically modified, and
wherein the biopolymer is selected from a C16 spider silk protein comprising the amino acid sequence of SEQ ID NO: 2, an S16 protein comprising the amino acid sequence of SEQ ID NO: 6; or a spinnable protein derived from these proteins having a sequence identity of at least about 60%.

39. The fibrous sheetlike structure of claim 38, wherein the fibrous sheetlike structure is obtained by means of a spinning process.

40. The fibrous sheetlike structure of claim 39, wherein the fibrous sheetlike structure is obtained by means of electrospinning of an electrospinnable solution which comprises at least one biopolymer and at least one active ingredient.

41. The fibrous sheetlike structure of claim 38, wherein the at least one active ingredient is in amorphous, semicrystalline or crystalline form.

42. The fibrous sheetlike structure of claim 38, wherein the active ingredient has been integrated into the carrier and/or adsorbed thereon.

43. The fibrous sheetlike structure of claim 38, wherein at least one active pharmaceutical ingredient is present.

44. The fibrous sheetlike structure of claim 43, wherein the active ingredient is an active cough-inducing and mucolytic ingredient.

45. The fibrous sheetlike structure of claim 44, wherein the active ingredient is guaiacol glyceryl ether or a derivative thereof.

46. The fibrous sheetlike structure of claim 38, wherein the active ingredient is an active crop protection ingredient.

47. The fibrous sheetlike structure of claim 38, wherein the active ingredient is an active skin- and/or hair-cosmetic ingredient.

48. The fibrous sheetlike structure of claim 38, wherein the carrier comprises at least one further polymer component which is a synthetic polymer.

49. The fibrous sheetlike structure of claim 48, wherein the synthetic polymer is a homo- or copolymer.

50. The fibrous sheetlike structure of claim 38, wherein the polymeric carrier is a composite polymer selected from the group consisting of
a) mixtures of at least 2 miscible biopolymers;
b) mixtures of at least 2 immiscible biopolymers;
c) mixtures of at least one synthetic homo- or copolymer and at least one biopolymer, which are miscible with one another; and
d) mixtures of at least one synthetic homo- or copolymer and at least one biopolymer, which are immiscible with one another.

51. The fibrous sheetlike structure of claim 48, wherein the synthetic polymer component has a molar mass in the range from about 500 to 10,000,000.

52. The fibrous sheetlike structure of claim 38, wherein the diameter of the active ingredient carrier fibers is 10 nm to 100 μm.

53. The fibrous sheetlike structure of claim 38, wherein the diameter of the active ingredient carrier fibers is 100 nm to 2 μm.

54. The fibrous sheetlike structure of claim 38, wherein the active ingredient loading is about 0.01 to 80% by weight, based on the solids content of the fibrous sheetlike structure.

55. The fibrous sheetlike structure of claim 38, selected from the group consisting of polymer fibers, polymer films and polymer nonwovens.

56. The fibrous sheetlike structure of claim 38, wherein carrier polymer components and active ingredients interact noncovalently.

57. An active ingredient-containing formulation comprising the fibrous sheetlike structure of claim 38 in processed form, optionally in combination with at least one further formulating aid.

58. The formulation of claim 57, comprising the fibrous sheetlike structure in comminuted or uncomminuted form.

59. The formulation of claim 57, comprising the fibrous sheetlike structure in compacted form, in powder form or applied to a carrier substrate.

60. The formulation of claim 57, selected from cosmetic, human and animal pharmaceutical, agrochemical formulations, food additives and animal feed additives.

61. A method for producing an active ingredient-containing formulation comprising utilizing the active ingredient containing fibrous sheetlike structure of claim 38.

62. A method for controlled release of an active ingredient comprising utilizing the formulation of claim 57.

63. A process for producing a fibrous sheetlike structure according to claim 38, comprising
a) mixing at least one active ingredient together with at least one biopolymer component in a combined liquid phase and
b) then embedding the active ingredient into the biopolymer fiber by means of spinning processes.

64. The process of claim 63, wherein at least one active ingredient and the biopolymer component are mixed in a solvent phase and spun from this mixture.

65. The process of claim 63, wherein at least one active ingredient and the biopolymer component are mixed in a mixture of at least two mutually miscible solvents, active ingredients and polymers being soluble at least in one of the solvents, and spun from this mixture.

66. The process of claim 63, wherein the biopolymer is an amphiphilic, self-assembly protein, which is mixed with at least one active ingredient in formic acid, and then spun from this mixture.

67. The process of claim 63, wherein the spinning process is an electrospinning process or a centrifuge (rotor) spinning process.

68. The process of claim 63, wherein the operating temperature is in the range from about 5 to 50° C.

69. The fibrous sheetlike structure of claim 38, which is essentially free of low molecular weight active ingredients.

70. A method of producing an active ingredient-containing or active ingredient-free formulation comprising combining the fibrous sheetlike structure of claim 69 with at least one further formulating aid.

71. The method of claim 70, wherein the formulation is selected from the group consisting of
(i) cosmetic,
(ii) human and animal pharmaceuticals,
(iii) agrochemical formulations, and
(iv) food and animal feed additives.

72. The fibrous sheetlike structure of claim 69, comprising a fibrous, polymeric, soluble and/or degradable carrier, wherein the carrier comprises, as a polymer component, at least one biopolymer which has optionally additionally been chemically and/or enzymatically modified, and wherein the biopolymer is an amphiphilic, self-assembly protein.

73. The fibrous sheetlike structure of claim 69, wherein the biopolymer is a silk protein selected from the R16 protein comprising the amino acid sequence of SEQ ID NO: 4, and the S16 protein comprising the amino acid sequence of SEQ ID NO: 6; or a spinnable protein derived from these proteins and having a sequence identity of at least about 60%.

74. A process for production of a wound care product and a hygiene article which comprises utilizing the fibrous sheetlike structure according to claim 71.

75. A wound care product comprising the fibrous sheetlike structure of claim 73.

76. A hygiene article comprising the fibrous sheetlike structure of claim 73.
